(19) United States
(12) Patent Application Publication
(10) Pub. No.: US 2005/0026957 A1
(43) Pub. Date: Feb. 3, 2005

(54) Methods of treating nodulocystic acne

(75) Inventors: John Sefton, Trabuco Canyon, CA (US); John R. Gibson, Laguna Beach, CA (US)

Correspondence Address: STOUT, UXA, BUYAN & MULLINS LLP, 4 VENTURE, SUITE 300, IRVINE, CA, 92618, US

(73) Assignee: Allergan, Inc., Irvine, CA (US)

(21) Appl. No.: 10/901,888

(22) Filed: Jul. 29, 2004

Related U.S. Application Data
(60) Provisional application No. 60/491,143, filed on Jul. 30, 2003.
(60) Provisional application No. 60/525,569, filed on Nov. 26, 2003.
(60) Provisional application No. 60/491,208, filed on Jul. 30, 2003.
(60) Provisional application No. 60/506,561, filed on Sep. 26, 2003.
(60) Provisional application No. 60/512,472, filed on Oct. 17, 2003.

(51) Int. Cl. A61K 31/4436

(52) U.S. Cl. 514/337

(57) ABSTRACT

Methods including orally administering retinoid components to a human or animal having nodulocystic acne to provide substantial reduction in nodulocystic acne in the human or animal and relatively little or substantially no reduction in sebum secretion in the human or animal relative to employing a reference retinoid agent, for example, a pan active retinoid agent, in an orally administering step using an amount of the reference retinoid agent to provide the same reduction in nodulocystic acne.

### RELATED APPLICATIONS

This application claims the benefit of Provisional U.S. Patent Application Ser. No. 60/491,143, filed Jul. 30, 2003, and application Ser. No. 60/525,569, filed Nov. 26, 2003.

Provisional U.S. Patent Application Ser. No. 60/491,208, filed Jul. 30, 2003, application Ser. No. 60/491,143, filed Jul. 30, 2003, application Ser. No. 60/506,561, filed Sep. 26, 2003, application Ser. No. 60/512,472, filed Oct. 17, 2003, and application Ser. No. 60/525,569, filed Nov. 26, 2003, in their entireties are hereby incorporated by reference.

### BACKGROUND OF THE INVENTION

The present invention relates to methods of providing therapeutic effects using retinoid components. More particularly, this invention relates to systemically administering to patients, that is humans or animals, without regard to the body weights of the patients, amounts of certain retinoids effective to provide reduction in the severity of various medical conditions, while, at the same time achieving one or more of consistent bioavailability, reduced drug interactions, and reduced side effects relative to administering a reference retinoid agent effective to provide the same therapeutic effect. In a further and more specific embodiment, the invention relates to orally administering to patients retinoid components selected from the group consisting of tazarotene, tazarotenic acid, derivatives of tazarotene, other precursors of tazarotenic acid, derivatives of tazarotenic acid and mixtures thereof in therapeutically effective amounts, for example amounts effective to reduce conditions such as psoriasis and nodulocystic acne, advantageously while resulting in one or more of the aforementioned advantages relative to a reference retinoid agent.

Retinoid drugs exert their therapeutic activity by acting as ligands, and therefore stimulating, activating blocking or inhibiting the biological activities, of either or both of the retinoid-associated nuclear receptors RAR (retinoic acid receptors) and RXR (retinoid X receptors). Although not wishing to be limited by any particular theory, each of these receptors is thought to undergo a conformational change when a cognitive agonist binds the receptor. This conformational change then results in the receptor stimulating or inhibiting the expression of a set of particular genes. This process is termed transactivation. In addition, there are myriad ligand-mediated effects, such as involvement in the stimulation or mediation of cellular phosphorylation cascades, which may not be transactivational events.

Also, the RAR and RXR receptors each have three major subtypes. RAR receptors comprise RAR alpha, RAR beta, and RAR gamma. Similarly, RXR receptors comprise RXR alpha, RXR beta, and RXR gamma.

A number of retinoid drugs are formulated for oral delivery. For instance, RAR agonists such as acitretin (Soriatane) and etretinate can be administered orally to treat psoriasis. RXR agonists such as bexarotene (Tagretin) can be administered orally to treat skin lymphoma. Tretinoin (Vesanoid), which binds and transactivates both RAR and RXR, can be administered orally to treat promoclocytic anemia, and isotretinoin (Accutane), which also affect both types of receptors, can be administered orally to treat acne.

A physician often takes a number of factors into account when prescribing any of the aforementioned oral retinoids. It is important, for example, to consider whether the bioavailability of the retinoid will be affected by the presence or absence of food in the patient's digestive tract. In the case of isotretinoin (Accutane), bexarotene (Targretin), and acitretin (Soriatane), the impact of food is well documented in that bioavailability is increased in the presence of food. See, for example, Colburn W. A. et al, J Clin Pharmacol. 1983; 23:534-539, hereby incorporated in its entirety herein by reference. For these retinoids, peak blood concentrations varied depending upon when the oral drug was administered relative to meals; however the time to peak blood concentration was not affected indicating that food increased the extent, but not the rate, of drug absorption. In the case of isotretinoin the total dose of the drug must be more than doubled to reach the same peak blood concentration in the fasted state as compared to following a high fat meal. This is seen as a significant disadvantage for these potent oral retinoids since the drug-absorption profile can drastically change depending upon the fasted or fed state of the patient.

Non-compliance with prescribed treatment regimens and systemic administration directions could undermine the effectiveness of these retinoids when treating disease states, such as, without limitation, for dermatological conditions e.g. psoriasis, acne; or for retinal ocular conditions e.g. age related macular degeneration, diabetic neuropathy and the like; for oncology applications, including treatment of dermatoses, melanomas, prostate cancer, as an adjunct to chemotherapy, for treatment of lung disorders such as emphysema and for treatment of other conditions responsive to retinoids. Moreover, retinoid absorption variability can lead not only to reduced therapeutic efficacy resulting from fluctuations of therapeutic drug-blood levels, but can also cause unwarranted drug side effects due to inadvertently high tissue exposure. It is therefore important, and indeed reinforced by prescribing physicians and the US Food and Drug Administration, that oral doses of retinoids be taken with food.

The prescribing physician should also consider the various side effects associated with different systemically administered retinoid drugs. The RAR agonists (acitretin, etretinate, and isotretinoin) are known to be associated with a large diversity of side effects at the doses necessary for acceptable or substantially optimal or optimal therapeutic activity, including, without limitation, side effects similar to those commonly associated with hypervitaminosis A, metabolic and nutritional side effects, whole body side effects, endocrine side effects, hemic and lymphatic system side effects, digestive system side effects, ocular side effects, cardiovascular side effects, nervous system side effects, psychiatric side effects, typical retinoid toxicity side effects, respiratory system side effects, ear side effects, gastrointestinal tract side effects, and urinary system side effects. The side effects associated with the use of these drugs are of considerable clinical significance and often preclude the use of these drugs in many patients or necessitate the close monitoring of liver enzymes, blood chemistries, etc.

In addition to the RAR agonists, RXR agonists, such as bexarotene, are also associated with many of the classic retinoid side effects, such as elevations of liver enzymes and blood lipids. Hypothyroidism also seems to be a relatively common feature of RXR-active retinoids and this condition is itself associated with many significant and serious complaints including mental confusion and depression.

Drugs such as tretinoin and isotretinoin that affect both RAR and RXR receptors are associated with both RAR and RXR-type side effects.

Retinoids are often formulated for topical administration to be therapeutically effective while reducing the occurrence and/or severity of side effects caused by systemic administration. Topical administration of retinoids results in reduced blood concentrations of the active drug, which can adversely impact the therapeutic effectiveness of the drug. For example, the maximum blood concentration of tazarotenic acid obtained by topical administration of tazarotene is often well less than 30 ng/ml.

Still another factor to be considered is the body weight of the patient for whom a retinoid is being prescribed. It has been established, for instance, that the bioavailability of RAR agonists such as acitretin, etretinate, and isotretinoin is increased with a reduced body weight. For these retinoids, bioavailability can drastically differ from patient to patient depending upon the body weight of each patient when a certain systemic, for example, oral, dose of the drug is administered.

In addition, the physician should consider what, if any, medications the patient is taking in addition to the retinoids, since certain of the aforementioned retinoids may be associated with drug interactions at the doses necessary for acceptable or substantially optimal or optimal therapeutic activity. The drug interactions associated with the use of these retinoids are often of considerable clinical significance. For example, it has been established that isotretinoin decreases blood concentrations of both ethinyl estradiol and norethindrone in coadministered contraceptive tablets and that acitretin interferes with the contraceptive effect of microdosed progestin “minipill” preparations. This is of particular clinical significance since retinoids have been identified as interfering with normal embryonic development leading to fetal malformations when administered during pregnancy.

Another factor that should be considered, especially in the treatment of acne, is the effect that a given retinoid has on the secretion of sebum in a patient. When retinoids, such as isotretinoin, are used to treat certain forms of acne, substantial reductions in sebum secretion occur. Sebum is secreted by the sebaceous glands and is a chemically complex oil that lubricates the skin and coats hair. In fact, published reports have linked the efficacy of isotretinoin in treating acne to its potential to inhibit sebaceous gland activity. In particular, such reports have concluded that the marked inhibitory effect of isotretinoin on sebaceous glands with a significant decrease in sebum secretion rate, for example, of about 90%, is certainly the main factor in the clinical response of severe acne with isotretinoin. See: Geiger, J. M.; Retinoids and Sebaceous Gland Activity, Dermatology, vol. 191, pps. 305-310 (1995); and Geiger, J. M. et al, Oral 13-cis Retinoic Acid is Superior to 9-cis Retinoic Acid in Sebosuppression in Human Beings, J. Am. Acad. Dermatology, vol. 34, pps. 513-515 (1996). Reducing sebum secretion can be detrimental to skin condition. For example, sebum is thought to provide a natural conditioning effect, keeping the skin smooth and supple and protecting against drying, scaling and itching.

Chandraratna U.S. Pat. No. 5,089,509, the disclosure of which is incorporated in its entirety herein by reference, discloses a group of compounds which may be used to treat acne and other dermatoses such as acne, Darier's disease, psoriasis, icthyosis, eczema, atopic dermatitis and epithelial cancers, as well as in treating arthritic diseases and other immunological disorders (e.g., lupus erythematosus), in promoting wound healing, in treating dry eye syndrome and in reversing the effects of sun damage to skin. Among the compounds disclosed by Chandraratna are the compounds known as tazarotene and tazarotenic acid. The patent discloses that when the retinoid-like compounds are used in the treatment of dermatoses, it will generally be preferred to administer the drug topically, though in certain cases such as treatment of severe cystic acne, oral administration may also be used.

Another patent of interest is Firestone et al U.S. Pat. No. 6,248,354, the disclosure of which is incorporated in its entirety herein by reference. The Firestone Patent discloses a capsule system for the oral delivery of an active agent, e.g., tazarotene, having low aqueous solubility and a vehicle for eliminating any need for initial active agent dissolution within the gastro-intestinal tract. The Firestone et al patent discloses that orally administered tazarotene to provide maximum blood level concentrations of tazarotenic acid in healthy subjects of between 5.24 and 44.3 ng/ml may be sufficient to effect the treatment of acne in a patient.

Neither the Chandraratna patent nor the Firestone et al patent specifically disclose the advantages of any of the disclosed compounds, such as tazarotene and tazarotenic acid, in reducing cystic acne, for example, to obtain specific therapeutic reductions in cystic acne, e.g., halting or arresting or inhibiting the progression of cystic acne, reducing or substantially eliminating one or more of the symptoms of cystic acne, reducing the size of one or more of the cystic acne lesions, reducing the number of the cystic acne lesions, substantial or complete curing of cystic acne, and the like. Moreover, neither patent specifically discloses the advantages of using such compounds at specific blood concentrations and/or for specific periods of time. Further, neither patent specifically discloses the advantages of using such compounds, for example, in reducing cystic acne, for example, as noted above, at specific daily doses and/or in specific dosage forms.

It would be advantageous to provide methods of administering retinoids to patients in amounts effective to provide desired therapeutic effects, while, at the same time, providing at least one other benefit, such as substantially constant or consistent bioavailability, reduced drug interactions, reduced side effects and the like, for example, relative to other retinoids.

### SUMMARY OF THE INVENTION

New therapeutic methods employing retinoid components have been discovered. The present methods involve systemic, preferably oral, administration to a human or animal of a retinoid component to provide a desired therapeutic effect.

The present methods are useful in providing desired therapeutic effects, including, without limitation, the treatment, preferably reduction, and prevention of acne, treatment and prevention of psoriasis, treatment and prevention of photodamage, treatment and prevention of skin disorders of keratinization, treatment and chemoprevention of cancer (e.g. skin cancer, prostate cancer, breast cancer, thyroid cancer, head and neck cancer, colon cancer, acute promyelocytic leukemia, cutaneous T-cell lymphoma), treatment and prevention of precancerous skin lesions e.g. actinic keratoses, treatment and prevention of emphysema, treatment and prevention of restenosis, treatment and prevention of atherosclerosis, treatment and prevention of macular degeneration, treatment and prevention of cervical dysplasia, and other conditions responsive to retinoids.

In general, the present invention is directed to methods for providing desired therapeutic effects to a human or animal which comprise systemically, preferably orally, administering to the human or animal a therapeutically effective amount of a retinoid component selected from active retinoid agents, precursors of active retinoid agents and mixtures thereof. The desired therapeutic effect advantageously is provided as a result of the administering step.

In one particularly useful embodiment, the administering is effective to provide for a maximum blood concentration of active retinoid agent in the human or animal of greater than 30 ng/ml or greater than 40 ng/ml or greater than 45 ng/ml or greater than about 50 ng/ml.

In one aspect of the invention, the orally administering step is effective to provide a substantially equivalent bioavailability of the retinoid component to the human or animal in the presence or absence of food in the gastrointestinal tract of the human or animal.

In an additional aspect of the invention, the orally administering step is effective to provide a substantially equivalent bioavailability of the retinoid component to the human or animal regardless of the body weight of the human or animal.

In a further aspect of the present invention, the orally administering step is effective to provide a more constant bioavailability of the retinoid component to a human or animal regardless of the body weight of the human or animal relative to employing a reference retinoid agent, such as isotretinoin, in place of the retinoid component in a substantially identical orally administering step, for example, in a human or animal of similar or substantially identical body weight.

In another aspect of the invention, the systemically administering step results in at least one fewer side effect or at least one reduced side effect relative to employing a reference retinoid agent, which reference agent preferably is selected from pan RAR-active retinoids, such as isotretinoin, acitretin, etretinate, tretinoin and the like, and RXR-active retinoids, for example, bexarotene and the like.

As used herein, the term “pan RAR-active retinoid” refers to a retinoid which affects RAR-alpha, RAR-beta and RAR-gamma substantially equally or non-selectively, i.e., where there is less than an about five-fold or less than an about ten-fold difference between the activity of the retinoid at each of the RAR alpha, RAR beta, and RAR gamma receptors.

In yet another aspect of the present invention, the systemically administering step results in at least one fewer or reduced drug interaction with another therapeutic agent being coadministered, for example, in the same composition or in separate compositions, relative to employing a reference retinoid agent in an identical systemically administering step to provide the same therapeutic effect, for example, at a dose effective to provide the same therapeutic effect. In one embodiment, the reference retinoid agent is selected from pan active retinoid agents and active retinoid agents effective to bind to RXR's.

The therapeutic agent being coadministered may include, without limitation, one or more of contraceptives, antibacterials, antifungals, antiparasitics, antivirals, antihistamines, decongestants, antiinflammatories, miotics, anesthetics, analgesics, chelating agents, antineoplastics, chemotherapeutic agents, antihypertensives, muscle relaxants, diagnostic agents, and mixtures thereof.

In a particularly useful embodiment, the invention comprises new methods for treating nodulocystic acne employing retinoid components. The present methods involve systemic, preferably oral, administration to a human or animal having nodulocystic acne of a retinoid component to provide the desired therapeutic effect, e.g., a reduction in nodulocystic acne, such as halting or arresting or inhibiting the progression of cystic acne, reducing or substantially eliminating one or more of the symptoms of cystic acne, reducing the size of one or more of the cystic acne lesions, reducing the number of the cystic acne lesions, substantial or complete curing of the cystic acne and the like, advantageously while reducing or even substantially eliminating the effect on sebum secretion resulting from such administration. In one embodiment, the systemic or oral administration of the retinoid component is effective to provide less reduction in sebum secretion in the human or animal relative to employing a reference retinoid agent in place of the retinoid component in a systemically or orally administering step using an amount of the reference retinoid agent to provide the same reduction in nodulocystic acne.

Among the advantages of reducing, or eliminating, the inhibitory effect on sebum secretion, in accordance with the present invention, are reduced incidences of dry skin (xerosis), scaling (desquamation) and itching relative to using other retinoids, such as isotretinoin, acitretin, etretinate, tretinoin, bexarotene and the like, to treat nodulocystic acne. Moreover, the present methods of effectively treating nodulocystic acne with a reduced effect on sebum secretion are quite unexpected in view of the prior use of isotretinoin to treat severe acne which is apparently based on a substantial reduction in sebum secretion.

In another aspect, the present invention is directed to methods for reducing, for example, as described elsewhere herein, nodulocystic acne, such as severe nodulocystic acne, in a human or animal which comprise systemically, preferably orally, administering to the human or animal having nodulocystic acne a therapeutically effective amount of a retinoid component selected from active retinoid agents, precursors of active retinoid agents and mixtures thereof, preferably tazarotene, tazarotenic acid, derivatives of tazarotene, other precursors of tazarotenic acid, derivatives of tazarotenic acid and mixtures thereof.

The administering, for example, the oral administering, step of the present invention advantageously is effective to provide a maximum blood or plasma concentration of an active retinoid agent in the human or animal of greater than 30 ng/ml or greater than 40 ng/ml or greater than 45 ng/ml or greater than about 50 ng/ml, and more preferably greater than about 60 ng/ml or greater than about 70 ng/ml or greater than about 80 ng/ml or greater than about 100 ng/ml. The desired therapeutic effect, e.g., a reduction in the nodulocystic acne, for example, as described elsewhere herein, advantageously is provided as a result of the administering step.

As used herein, the term “derivative” refers to a compound or other substance which is sufficiently structurally similar to the compound or other substance of which it is a derivative to have substantially the same or similar usefulness or efficacy, for example, as an active retinoid agent or a precursor of an active retinoid agent, as the compound or other substance of which it is a derivative. Examples of useful derivatives often include, without limitation, biocompatible salts, esters, hydrates and the like, of a compound or other substance.

As used herein, the term “precursors of active retinoid agents” means compounds or other substances which can be metabolized, converted or formed, for example, after being ingested or introduced into a body of a human or animal, into active retinoid agents. For example, tazarotene and one or more derivatives of tazarotene can be considered precursors of active retinoid agents because tazarotene and one or more of its derivatives, after ingestion or introduction into the body of a human or animal, are converted into tazarotenic acid, an active retinoid agent, or one or more derivatives of tazarotenic acid, also active retinoid agents. In certain cases, a derivative of an active retinoid agent may be a precursor of an active retinoid agent and/or vice versa.

In one aspect of the invention, the systemically administering step results in or is conducted at conditions effective to provide less reduction in sebum secretion in the human or animal relative to employing a reference retinoid agent. The reference retinoid agent preferably is selected from pan RAR-active retinoids such as isotretinoin, and RXR-active retinoids, for example, bexarotene and the like. The systemically administering step using an amount of one of the reference retinoid agent is effective to provide the same reduction, for example, as described elsewhere herein, in nodulocystic acne as the present systemically administering step.

In another aspect of the present invention, the retinoid component is selected from active RAR agents or agonists which are substantially ineffective to bind to or activate RXRs, precursors of active RAR agents or agonists which are substantially ineffective in binding to or activating RXRs and mixtures thereof.

In one embodiment, the systemically administering step of the present methods is effective in providing the desired therapeutic effect, and results in or is conducted at conditions effective to provide less reduction in sebum secretion in the human or animal relative to employing a RXR active retinoid agent which is effective in binding to RXRs in place of the retinoid component in a systemically administering step at a dose of the RXR active agent, or using an amount of the RXR active agent, effective to provide the same therapeutic effect, for example, the same reduction in the nodulocystic acne.

In a further aspect of the present invention, the retinoid component is selected from active RAR agonists effective to selectively, or even specifically, affect, for example, activate, at least one, and preferably both, of RAR-beta and RAR-gamma relative to RAR-alpha, precursors of such active RAR agonists and mixtures thereof. As used in this context, the term “selectively” means that the presently useful RAR agonists precursors of RAR agonists and mixtures thereof are more effective, preferably at least about 10 or about 100 times to about 1000 times or more as effective, to affect times at least one, and preferably both, of RAR-beta and RAR-gamma relative to RAR-alpha. The systemically administering step is effective to provide the desired therapeutic effect, e.g., a reduction in the nodulocystic acne, and is conducted at conditions effective to result in or to provide less reduction in sebum secretion in the human or animal relative to employing a pan active or substantially non-selective RAR retinoid agent, such as described elsewhere herein, in place of the retinoid component in a systemically administering step using an amount of the pan active or substantially non-selective RAR retinoid agent to provide the same therapeutic effect, that is the same reduction in the nodulocystic acne.

The present methods advantageously provide substantial reductions, as described elsewhere herein, in nodulocystic acne. Preferably, nodulocystic acne reductions of at least about 60% or at least about 70% or at least about 80% or at least about 85% or at least about 90% are provided, with less reduction in sebum secretion, as described elsewhere herein.

Administration, e.g., systemically, preferably orally, administering, of the presently useful retinoid components often occurs for a period of time in excess of about 1 week, preferably in excess of about 4 weeks, or in excess of about 6 weeks, or in excess of about 12 weeks or in excess of about 20 weeks. Daily doses of the retinoid component can vary over a wide range. In one embodiment, at least about 0.75 mg or at least about 1 mg or at least about 1.5 mg or at least about 3.0 mg or at least about 5 mg or about 6 mg or more of the retinoid component are administered to the human or animal on a daily basis. In one embodiment, the daily dose advantageously is in a range of about 1 mg to about 6 mg of the retinoid component.

In a very useful embodiment, the administering step comprises orally administering a capsule, for example, a hard gel capsule or a soft gel capsule, containing the retinoid component to the human or animal. Among the capsule systems useful in accordance with the present invention are those disclosed in Firestone et al U.S. Pat. No. 6,248,354. Firestone et al discloses capsules, for example, soft gelatin capsules, with the following fill formulations:

| | | Concentration (mg/capsule) | |
|---|---|---|---|
| | | 0.7 mg Soft | 0.2 mg Soft |
| | | Gelatin | Gelatin |
| | | Capsule | Capsule |
| Ingredient | Function | (9096X) | (9154X) |
| Fill Formulation: | | | |
| Tazarotene | Active | 0.70 | 0.20 |
| Butylated | Anti-oxidant | 0.05 | 0.05 |
| Hydroxyanisole NF | | | |
| Sorbitan | Emulsifier | 5.0 | 5.0 |
| Monooleate NF | | | |
| Polysorbate 80 NF | Co-emulifier | 0.25 | 0.25 |
| Medium-chain | Lipophilic | 94.0 | 94.5 |
| Triglycerides EP | vehicle | | |

Of course, other formulations can be effectively used for oral administration of the presently useful retinoid components. Also, the formulations including the presently useful retinoid components may be chosen or selected depending, for example, on the mode of systemic administration of the composition. For example, and without limitation, formulations for oral administration, transdermal administration, rectal (suppository) administration, administration by injection and other non-oral administrations advantageously have different chemical make-ups, one from the other. This is so in order to provide a formulation which has highly suitable properties to facilitate the mode of administration chosen. Different formulations for use in the same mode of administration may be employed, for example, to effectively or more effectively meet the needs and/or requirements of the patient and/or the application involved. For example, and without limitation, formulations for oral administration can be in forms including soft capsules, hard capsules, powers, pills, tablets, liquids, syrups, elixirs and the like and mixtures or combinations thereof.

The selection of a retinoid component useful in accordance with the present invention can be accomplished using straightforward, conventional testing and/or assays, such as the transactivation assays set forth in Evans et al., U.S. Pat. Nos., 5,217,867; 5,262,300; 5,310,662; and 5,906,920, each of which is hereby incorporated in its entirety herein by reference, which are well known in the art. Such testing and/or assays can identify suitable useful retinoid components without undue experimentation.

Each and every feature described herein, and each and every combination of two or more of such features, is included within the scope of the present invention provided that the features included in such a combination are not mutually inconsistent.

These and other aspects and advantages of the present invention are set forth in the following detailed description, examples and claims.

### DETAILED DESCRIPTION

The present methods provide desired therapeutic effects employing certain retinoid components, particularly when administered systemically, for example, orally, to provide at least one desired therapeutic effect, and to advantageously result in one or more of the following: reduced side effects, reduced drug interactions, increased and/or substantially constant or consistent bioavailability and the like, for example, relative to systemically administering a reference retinoid agent effective to provide the same therapeutic effect or effects.

In one embodiment, the present methods provide that the bioavailability of the presently preferred retinoid components, when orally administered, is relatively or substantially unaffected by the presence/absence of food in the gastrointestinal tract, for example, the upper gastrointestinal tract, of the patient.

In one very useful embodiment, the administering step is repeated at different times without regard to whether or not food is substantially simultaneously ingested by the human or animal, or when the human or animal being treated has last eaten or is eating. This feature of the present invention provides substantial flexibility as to under what conditions the retinoid component is administered. Fewer restrictions are required so that the regimen under which the retinoid component is prescribed and administered is substantially simplified. Such a more flexible or less restrictive regimen in accordance with the present invention provides for enhanced patient compliance with the regimen. This is a substantial advantage of the present invention.

The reduced dependence of bioavailability of a retinoid component on the presence or absence of food provides for allowing the administering step to be conducted at least once with substantially simultaneous ingestion of food by the human or animal and at least once without substantially simultaneous ingestion of food by the human or animal, for example, with substantially similar blood concentrations of the drug being achieved each time.

In one embodiment, the present invention provides methods in which the bioavailability of certain retinoid components, when orally administered, is relatively unaffected by the body weight of the patient. For example, oral administration of the presently useful retinoid components may advantageously achieve substantially equivalent drug bioavailability regardless of body weight of the patient, for example, based on human pharmacokinetic parameters maximum concentration (Cₘₐₓ) and Area under the concentration-time Curve (AUC), or a more constant or consistent drug bioavailability relative to other or reference active retinoid agents, such as isotretinoin, the bioavailability of which is substantially affected by the body weight of the patient. This substantially equivalent or more constant or consistent bioavailability regardless of body weight feature of the present methods provides the treating physician with substantial flexibility and substantial elimination of concerns with regard to adjusting dosage to take into account patient body weight. A single dose form, that is a dose form having a single fixed or standard amount of the retinoid component, can be prescribed regardless of the body weight of the patient. This “single dose” feature of the present invention may lead to a more simple and straightforward, yet effective, treatment regimen with better patient compliance. Using the present invention may also provide additional benefits such as, enhanced therapeutic benefits, and reduced incidence and/or severity of side effects.

The present orally administering step advantageously is effective to provide a more constant or consistent bioavailability or a substantially equivalent bioavailability of the retinoid component to a human or animal regardless of the body weight of the human or animal.

The systemically, preferably orally, administering step of the present methods preferably is effective to provide a bioavailability of the retinoid component to the human or animal differing by less than about 70%, preferably by less than about 50%, more preferably by less than about 30%, and still more preferably by less than about 15%, regardless of the body weight of the human or animal, for example, when a certain dosage form which includes a same given therapeutic amount of retinoid component is administered to humans or animals of differing body weights, for example, differing body weights ranging from about 40 kg to about 130 kg, in the same amount of time. Such relative independence of the bioavailability of the retinoid component with regard to patient body weight is advantageously increased relative to the use of various commercially available oral retinoids, for example, isotretinoin, bexarotene and acitretin.

For the purposes of this application, the bioavailability of a drug may be based on the human pharmokinetic parameters of maximum blood concentration (Cₘₐₓ) and Area under the blood concentration-time curve (AUC). For example, a drug is said to have substantially equivalent bioavailability in the fasted state, that is after an 8-10 hour fast (being without food), and in the fed state, that is the drug is administered to a patient within 30 minutes after the patient consumes a high fat meal, if the drug exhibits a lack of food effect as defined by the U.S. Food and Drug Administration. For example, such substantially equivalent bioavailability is present if a drug exhibits substantially the same Cₘₐₓ and AUC when orally administered in both the fasted state and the fed state, or when orally administered to patients of differing body weights.

One way of determining bioavailability of an active retinoid component is to compare the values of Cₘₐₓ and AUC for the same retinoid component when taken in the presence (fed state) and absence (fasted state) of food or when taken by patients of differing body weights. If the values of Cₘₐₓ and AUC are substantially the same, for example, in both the fed and fasted states or in the patients of differing body weights, or if those values are more constant relative to a reference active retinoid agent, such as isotretinoin, the bioavailability of which is substantially affected by the presence or absence of food or body weight, then the active retinoid component is said to have a more constant bioavailability in the presence or absence of food, or regardless of body weight, or to have a more consistent bioavailability in the presence or absence of food, or regardless of body weight, relative to the reference retinoid agent.

In one embodiment, the retinoid component is said to have substantially equivalent bioavailability regardless of body weight if Cₘₐₓ and AUC are substantially the same, for example, within about 15% or within about 30% or within about 50%, regardless of whether the retinoid component is administered in the presence or absence of food (in the fed or fasted state), or for a number of patients having different body weights ranging from about 40 kg to about 130 kg, all of whom who have been given the same dosage of the retinoid component under identical administering circumstances and conditions.

This substantial food/drug bioavailability or absorption independence of the present methods provides the patient with substantial flexibility and substantial elimination of concerns as to whether dose administration should be before, with, or after food consumption. In addition, because the bioavailability of the drug is substantially unaffected by body weight, the physician can be more flexible in treating the patient, and need not take body weight into account when determining the dosage.

In one embodiment, methods are included for providing desired therapeutic effects, preferably the same therapeutic effect, to a plurality of humans or animals having differing body weights. Such methods include providing a plurality of dosage forms each of which has the same therapeutically effective amount of a retinoid component, as described herein. The same number of the dosage forms is orally administered to each of the plurality of humans or animals in the same amount of time. Such oral administration provides the desired therapeutic effect to each of the plurality of humans or animals. In addition, such oral administration provides a substantially equivalent bioavailability, or a more constant or consistent bioavailability, as described herein, of the retinoid component to each of the plurality of humans or animals.

In one embodiment, a single dose form, that is a dose form having a single fixed or standard amount of the retinoid component, can be prescribed regardless of the weight of the patient.

One or both of such features, for example, prescribing and using a single dose form regardless of body weight and the freedom to take the medication independently of meals, that is with or without food, lead to simpler, less restrictive, and straightforward, yet effective, treatment regimens with better patient compliance.

The relative independence of the bioavailability of the retinoid components used in accordance with the present invention regardless of body weight and in the presence or absence of food is advantageously increased relative to the use of various commercially available oral retinoids, for example, isotretinoin, bexarotene and acitretin, all of which show a substantial variation due to body weight and/or the presence/absence of food.

The systemically, preferably orally, administering step advantageously is effective to provide a more constant bioavailability or a substantial equivalent biocompatibility of the retinoid component to the human or animal regardless of body weight, and in the presence or absence of food, for example, in the upper gastrointestinal tract of a human or animal. The administering step is advantageously effective to provide a more constant bioavailability or a substantially equivalent biocompatibility of the retinoid component to a human or animal regardless of body weight and in the presence or absence of substantially undigested food or partially digested food in a human or animal, for example, in the upper gastrointestinal tract of the human or animal.

The administering step is further effective to reduce and/or eliminate one or more disadvantageous interactions with substances such as therapeutic components or drugs being coadministered, and/or to result in reduced incidence and/or severity of one or more side effects relative to other retinoid agents, as described herein. Such reduced side effects and/or drug interactions facilitate the use of retinoid components in accordance with the present invention to effectively provide the desired therapeutic effect without subjecting the patient to side effects or the severity of side effects previously associated with retinoid active agents, and/or with reduced concern that the patient is being exposed to risks of one or more detrimental drug interactions.

Among the side effects that can be reduced in severity or substantially eliminated in accordance with the present invention include, but are not limited to, metabolic and nutritional side effects, whole body side effects, endocrine side effects, hemic and lymphatic system side effects, digestive system side effects, ocular side effects, cardiovascular side effects, nervous system side effects, psychiatric side effects, typical retinoid toxicity side effects, respiratory system side effects, ear side effects, gastrointestinal tract side effects, urinary system side effects and the like.

The following are more specific examples of side effects which may be mitigated against in accordance with the present invention.
Typical Retinoid Toxicity: this side effect is similar to that in patients taking high doses of vitamin A and includes headache, fever, skin mucous membrane dryness, bone pain, nausea/vomiting, rash, mucositis, pruritus, increased sweating, visual disturbances, ocular disorders, alopecia, skin changes, changed visual acuity, bone inflammation, and visual field defects.
RA-APL Syndrome: characterized by fever, dyspnea, weight gain, radiographic pulmonary infiltrates and pleural or pericardial effusions. This syndrome is occasionally accompanied by impaired myocardial contractility and episodic hypotension and is observed with or without concomitant leukocytosis.
Body as a Whole: general disorders includes malaise, shivering, hemorrhage, infections, peripheral edema, pain, chest discomfort, edema, disseminated intravascular coagulation, weight increase, injection site reactions, anorexia, weight decrease, myalgia, flank pain, cellulitis, face edema, fluid imbalance, pallor, lymph disorders, acidosis, hypothermia, and ascites.
Respiratory System Disorders: include upper respiratory tract disorders, dyspnea, respiratory insufficiency, pleural effusion, pneumonia, rales, expiratory wheezing, lower respiratory tract disorders, pulmonary infiltration, bronchial asthma, pulmonary edema, larynx edema, and unspecified pulmonary disease.
Ear Disorders: ear disorders are consistently reported, with earache or feeling of fullness in the ears also reported. Hearing loss or other unspecified auricular disorders are observed, with infrequent reports of irreversible hearing loss.
Gastrointestinal Tract (GI) Disorders: include GI hemorrhage, abdominal pain, other gastrointestinal tract disorders, diarrhea, constipation, dyspepsia, abdominal distention, hepatosplenomegaly, hepatitis, ulcer, and unspecified liver disorder.
Cardiovascular and Heart Rate and Rhythm Side Effects: arrhythmias, flushing, hypotension, hypertension, phlebitis, cardiac failure, cardiac arrest, myocardial infarction, enlarged heart, heart murmur, ischemia, stroke, myocarditis, pericarditis, pulmonary hypertension, and secondary cardiomyopathy.
Central and Peripheral Nervous System Disorders and Psychiatric Side Effects: dizziness, paresthesias, anxiety, insomnia, depression, confusion, cerebral hemorrhage, intracranial hypertension, agitation, hallucination, abnormal gait, agnosia, aphasia, asterixis, cerebellar edema, cerebellar disorders, convulsions, coma, CNS depression, dysarthria, encephalopathy, facial paralysis, hemiplegia, hyporeflexia, hypotaxia, no light reflex, neurologic reaction, spinal cord disorder, tremor, leg weakness, unconsciousness, dementia, forgetfulness, somnolence, and slow speech.
Urinary System Disorders: renal insufficiency, dysuria, acute renal failure, micturition frequency, renal tubular necrosis, and enlarged prostate.

The use of oral retinoids has been implicated in severe psychiatric side effects, such as depression, including but not limited to, severe and/or chronic depression, for example, leading to suicidal thoughts, suicide attempts and even suicides. The presently useful retinoid components, when orally administered in accordance with the present invention, provide the desired therapeutic effect while substantially reducing the severity and/or occurrence of one or more of such severe psychiatric side effects.

Drug interactions that are reduced in severity or substantially eliminated in accordance with the present invention include drug interactions with contraceptives, such as those interactions where the effectiveness of a contraceptive is reduced. For example, it has been established that certain retinoids, such as acitretin, interfere with the contraceptive effect of microdosed progestin preparations. Coadministration of bexarotene with tamoxifen, an anti-breast cancer medication, results in a reduced plasma concentration of tamoxifen in patients relative to the plasma concentration of tamoxifen in patients administered tamoxifen in the absence of bexarotene. This drug interaction may be mediated through an induction of P450 3A4. Based on this known interaction, bexarotene, an RXR active agent, may increase the rate of metabolism and reduce the plasma concentrations of other substances metabolized by P450 3A4, including hormonal contraceptives.

Examples of contraceptives of particular interest for use as described herein include contraceptives which comprise one or more hormones, one or more hormone derivatives or mixtures thereof, such as estrogen-based contraceptives, progestin-based contraceptives and the like. Contraceptives for use as described herein include, without limitation, one or more of norethindrone, ethinyl estradiol, norgestimate, levonorgestrel, deacetyl norgestimate and mixtures thereof. Certain name brand contraceptives contemplated for use in accordance with the present invention include, without limitation, Ortho-Novum® and Ortho TriCyclen®.

Examples of other substances which are substantially unaffected by coadministration of the presently useful compounds are anti-inflammatories, such as cortisone, hydrocortisone, hydrocortisone esters, betamethasone, dexamethasone, dexamethasone sodium phosphate, prednisone, methylprednisolone, medrysone, fluorometholone, prednisolone, prednisolone sodium phosphate, triamcinolone, indomethacin, sulindac, its salts and its corresponding sulfides, analogs thereof and the like; non-steroidal, anti-inflammatory substances, such as acetylsalicylic acid (aspirin), indomethacin, diclofenac, fenoprofin, ketorolac tromethamine, diclofenac sodium, suprofen and the like; antimicrobial agents including antibacterial agents and antifungal agents, such as tetracyclines, aminoglycosides, vancomycin, cephlosporins, sulfonamides, loridine (cephaloridine), chloramphenicol, clindamycin, amikacin, tobramycin, methicillin, lincomycin, oxycillin, penicillin, amphotericin B, polymyxin B, cephalosporin family agents, ampicillin, bacitracin, carbenicillin, cepholothin, colistin, erythromycin, streptomycin, neomycin, sulfacetamide, silver nitrate, sulfisoxazole and diolamine, beta-lactam antibiotics, such as cefoxitin, n-formamidoylthienamycin and other thienamycin derivatives, tetracyclines, chloramphenicol, neomycin, carbenicillin, colistin, penicillin G, polymyxin B, vancomycin, cefazolin, cephaloridine, chibrorifamycin, gramicidin, bacitracin and sulfonamides, aminoglycoside antibiotics such as gentamycin, kanamycin, amikacin, sisomicin and tobramycin, nalidixic acid and its analogs such as norfloxacin and the antimicrobial combination fluoroalanine/pentizidone, nitrofurazones, nystatin, flucytosine, natamycin and miconazole, fluoroquinolones, analogs thereof and the like; antiparasitic compounds and/or anti-protozoal compounds, such as ivermectin, pyrimethamine, trisulfapidimidine, clindamycin and corticosteroid preparations and the like; compounds having antiviral activity, such as acyclovir, 5-iodo-2′-deoxyuridine (IDU), adenosine arabinoside (Ara-A), trifluorothymidine, interferon, and interferon-inducing agents such as poly I:C, idoxuridine, trifluorouridine, vidarabine (adenine arabinoside), acyclovir (acycloguanosine), gancyclovir, pyrimethamine, trisulfapyrimidine-2, clindamycin, nystatin, flucytosine, natamycin, miconazole and piperazine derivatives, for example, diethylcarbamazine, and the like.

Examples of other substances which are substantially unaffected by coadministration of the presently useful compounds are NMDA antagonists, antihistaminics and decongestants, such as pyrilamine, chlorpheniramine, tetrahydrazoline, antazoline, analogs thereof and the like; mast-cell inhibitors of histamine release, such as cromolyn, miotics and anticholinergics such as echothiophate, physostigmine salicylate, diisopropylfluorophosphate, epinephrine, dipivaloylepinephrine, neostigmine echothiopate iodide, demecarim bromide, carbamoyl choline chloride, methacholine, bethanechol analogs thereof and the like; mydriatics, such as atrophine, homatropine, scopolamine, hydroxyamphetamine, ephedrine, cocaine, tropicamide, phenylephrine, cyclopentolate, oxyphenonium, eucatropine and the like; adrenergic agonists and/or antagonists such as epinephrine and epinephrine complexes, and prodrugs and the like; carbonic anhydrase inhibitors, such as acetazolamide, dichlorphenamide, 2-(p-hydroxyphenyl)-thiothiophene-sulfonamide, 6-hydroxy-2-benzothiazolesulfonamide, 6-pivaloyloxy-2-benzothiazolesulfonamide and the like; anesthetic agents, such as etidocaine, cocaine, benoxinate, dibucaine hydrochloride, dyclonine hydrochloride, naepaine, phenacaine hydrochloride, piperocaine, proparacaine hydrochloride, tetracaine hydrochloride, hexylcaine, bupivacaine, lidocaine, mepivacaine, prilocaine and the like; ophthalmic diagnostic agents, such as (a) those used to examine the retina such as sodium fluorescein, (b) those used to examine the conjunctiva, cornea and lacrimal apparatus, such as fluorescein and rose bengal and (c) adrenaline, atropine, hydroxyamphetamine and the like; ophthalmic agents used as adjuncts in surgery, such as alpha-chymotrypsin, hyaluronidase and the like; chelating agents, such as ethylenediaminetetraacetic acid (EDTA), deferoxamine and the like; immunosuppressants and anti-metabolites, such as methotrexate, cyclophosphamide, 6-mercaptopurine, azathioprine and the like; and combinations of the agents mentioned above, such as antibiotics/antiinflammatories combinations, for example the combination of neomycin sulfate and dexamethasone sodium phosphate, and the like.

Examples of other substances which are substantially unaffected by coadministration of the presently useful compounds are mitotics, such as pilocarpine, acetylcholine chloride, isoflurophate, demacarium bromide, echothiophate iodide, phospholine iodide, carbachol, physostigimine, epinephrine and salts, such as dipivefrin hydrochloride, and dichlorphenamide, acetazolamide, methazolamide and the like; anti-cataract and anti-diabetic retinopathy substances, such as aldose reductase inhibitors, such as tolrestat, lisinopril, enalapril, and statil and the like; thiol cross-linking substances; anti-clotting substances, such as tissue plasminogen activator, urokinase, and streptokinase and the like; anti-tissue damage substances, such as superoxide dismutase, proteins and nucleic acids, such as mono- and polyclonal antibodies, enyzmes, protein hormones and genes encoding the same, gene fragments and plasmids and the like; cycloplegic and mydriatic substances, such as atropine, cyclogel, scopolamine, homatropine mydriacyl and the like.

Examples of other substances which are substantially unaffected by coadministration of the presently useful compounds are anti-tumor substances, such as antineoplastics, chemotherapeutic agents and pharmaceutically acceptable salts thereof, for example, leucovorin, antimetabolites, 6-mercaptopurine, methotrexate, 5-fluorouracil, anthracyclines, doxorubicin, daunorubicin, mitoxantrons and the like. Also included are bleomycin, nitrosoureas, for example, carmustine (BCNU), procarbazine, vincriotine, thiotepa, fluoxymesterone, vinblastine, etopside, decarbazine, levamisole, irinotecan, mitomicin-C, streptozocin and the like; camptothcen (CPT) drugs; estrogen receptor antagonists; anti-cancer substances, such as methotrexate, adriamycin, bleomycin, triamcinolone, mitomycin, cis-platinum, vincristine, vinblastine, actinomycin-D, ara-c, bisantrene, CCNU, activated cytoxan, DTIC, HMM, melphalan, mithramycin, procarbazine, VM26, VP16, tamoxifen and the like; immune modulators, other than those indicated previously, and biological cancer theraputic agents, such as p53 genes, antibodies, interferons, interlukens, hematopoietic growth factors, tumor necrosis factors, gene therapy agents containing genetic material and the like.

In one useful embodiment, the systemically, preferably orally, administering is effective to provide a maximum plasma or blood concentration of active retinoid agent in the human or animal of greater than 30 ng/ml, preferably greater than 40 ng/ml or greater than about 45 ng/ml or greater than about 60 ng/ml or greater than about 70 ng/ml or greater than about 80 ng/ml, for example, greater than about 100 ng/ml. Of course, the concentration of active retinoid agent in the blood of the human or animal should be therapeutically effective and should be less than that which would cause substantial harm or be toxic to the patient. Because of the reduction in the incidence and/or severity of side effects and/or drug interactions in accordance with the present methods, increased maximum blood concentrations of the presently useful retinoid components, relative to the maximum blood concentration of a reference retinoid agent, may be employed to the therapeutic advantage of the human or animal while still resulting in reduced risk of side effects and/or drug interactions. This is an important advantage of the present invention.

Concentration of a substance, for example, a retinoid, in blood may be determined using a liquid chromatography-mass spectroscopy-mass spectroscopy (LC-MS/MS). In pharmaceutical applications, drug concentrations are typically reported in terms of blood plasma concentration rather than whole blood concentration. Thus, for the purposes of this application, references to “blood concentration” may be understood to mean “blood plasma concentration.”

The systemically administering advantageously comprises other than topically administering to the human or animal the retinoid component. Preferably, although not exclusively, the administering comprises a step selected from the group consisting of orally administering to the human or animal the retinoid component, transdermally administering to the human or animal the retinoid component, intravenously administering to the human or animal the retinoid component, subcutaneously administering to the human or animal the retinoid component, intramuscularly administering to the human or animal the retinoid component, intraperitoneally administering to the human or animal the retinoid component, rectally administering to the human or animal the retinoid component, one or more of like administering steps and combinations thereof. In a very useful embodiment, the administering comprises systemically, preferably orally, administering to the human or animal the retinoid component. Advantageously, the retinoid component is not topically administered to the skin of the human or animal in an amount effective to treat the patient's condition while, or during the time, the retinoid component is being systemically administered to the human or animal, for instance, to treat the same condition.

In one embodiment, the systemically administering step is effective to provide an increased blood concentration of active retinoid agent in the human or animal relative to topically administering an identical amount of the retinoid component to the human or animal.

The retinoid component preferably includes an active retinoid agent and/or a precursor of an active retinoid agent effective to selectively, and even specifically, affect, for example, bind to and/or activate and/or inhibit the activation of and/or block, at least one of RAR-beta and RAR-gamma relative to RAR-alpha.

As used herein, the terms “selectively” or “more selectively” refer to the ability of an active retinoid agent to affect RAR-beta and RAR-gamma relative to RAR-alpha. In preferred embodiments, the presently useful active retinoid agents affect RAR-beta and RAR-gamma at least about 5 times, at least about 10 times, at least about 20 times, at least about 50 times, at least about 100 times, or about 1000 times more than RAR-alpha. The term “specifically” refers to the ability of an active retinoid agent to affect RAR-beta and RAR-gamma without substantially affecting, or preferably without affecting in a detectable way, RAR alpha.

In one embodiment, the retinoid component includes an active retinoid agent or a precursor of an active retinoid agent effective to selectively or even specifically affect both RAR-beta and RAR-gamma relative to RAR-alpha. The retinoid component advantageously includes an active retinoid agent or a precursor of an active retinoid agent effective to selectively or even specifically activate or inhibit the activation of or block at least one or both of RAR-beta and RAR-gamma relative to RAR-alpha. In one embodiment, the retinoid component includes an active retinoid agent or a precursor of an active retinoid agent effective to selectively or even specifically activate at least one of or both RAR-beta and RAR-gamma relative to RAR-alpha.

Although the present invention is applicable to a large variety of retinoid components, such as active retinoid agents or precursors of active retinoid agents which have RAR-antagonist activity and RAR-inverse agonist activity, the present invention is particularly useful with retinoid components which include active retinoid agents or precursors of active retinoid agents which have RAR-agonist activity.

In one useful embodiment, the retinoid component includes an active retinoid agent having a substantial degree of water solubility. For example, an active retinoid agent may be more water soluble than isotretinoin, or may be converted, for example, metabolically converted, in the human or animal into an active retinoid agent having a substantial degree of water solubility, e.g., into an active retinoid agent more water soluble than isotretinoin. In this way, it is possible to design the active retinoid agent to avoid having the active agent cross lipid barriers, such as the blood brain barrier and the retinal-blood barrier.

Advantageously, the retinoid component comprises an active RAR ligand which is substantially ineffective to bind to or activate or block RXRs and/or a precursor of an active RAR ligand substantially ineffective to bind to or activate or block RXRs.

In a broad sense, any compound can be tested for RAR activity, for example, using conventional and well known techniques, for example, without limitation, those described in the above-noted patents, each of which is incorporated in its entirety herein by reference. Once a compound has been determined to have suitable RAR activity, it can be administered to a test animal, such as with and without simultaneous ingestion of food, for example, in the fed and fasted states, and/or with appropriate monitoring of body weight, and/or with appropriate monitoring for drug interactions and/or side effects and/or efficacy with regard to reducing nodulocystic acne. Comparing the results of such administering and/or monitoring with similar administering and/or monitoring of test animals given reference retinoid agents allows one to determine if the compound is useful in accordance with the present invention.

In other aspects of the present invention, one or more compounds, for example, from a screening library of compounds, which are known to have or have been tested, using conventional and well known techniques, and found to have useful RAR activity, can be individually or collectively tested for RXR activity using conventional and well known testing procedures. See, for example, the above-noted Evans et al. patents, in particular U.S. Pat. No. 5,906,920.

Compounds with substantially no RXR activity can be selected for further testing. Compounds with desired RAR activity and substantially no RXR activity are useful in accordance with one or more aspects of the present invention.

Other well known and straightforward test methods and/or assays may be employed to determine the selectivity or specificity of an RAR active compound to RAR-alpha, RAR-beta and RAR-gamma. For example, using conventional and well known assays, for example, such as set forth in Klein et al. U.S. Pat. No. 5,776,699, the disclosure of which is incorporated in its entirety herein by reference, and/or the above-noted Evans et al. patents, the selectively or specificity of a compound to RAR-alpha, RAR-beta and RAR-gamma can be determined. Based on the results of such assays, one can determine whether or not a compound is useful in accordance with one or more aspects of the present invention.

Further confirmation that any compound is useful in accordance with the present invention can be obtained by systemically, preferably orally, administering the compound to an animal in the fed and fasted states and comparing pharmacokinetic data, or administering the compound to a number of animals of differing body weights and comparing pharmacokinetic data, or by administering the compound to an animal (or series of animals) and monitoring for side effects and/or the presence or absence of interactions with substances, for example, therapeutic components being coadministered, and/or for efficacy with regard to reducing nodulocystic acne.

In any event, determining which compounds are useful in accordance with the present invention can be accomplished using conventional and well known techniques, without undue experimentation.

Some examples of structures and methods of making preferred retinoid components, are provided in U.S. Pat. No. 5,776,699, U.S. Pat. No. 5,958,954, U.S. Pat. No. 5,877,207, and U.S. Pat. No. 5,919,970 which are all incorporated by reference herein in their entireties. Many of the following compounds are included in one or more of these patents.

Among the retinoid components useful in the present invention include the following compounds of formula I 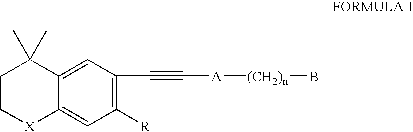
wherein X is S, O, or NR═ where R═ is hydrogen or lower alkyl; R is hydrogen or lower alkyl; A is pyridinyl, thienyl, furyl, pyridazinyl, pyrimidinyl or pyrazinyl; n is 0-2; and B is H, —COOH or a pharmaceutically acceptable salt, ester or amide thereof, —CH₂OH or an ether or ester derivative, or —CHO or an acetal derivative, or —COR₁ or a ketal derivative where R₁ is —(—CH₂)ₘCH₃ where m is 0-4.

The compounds of formula I can be made by reacting a compound of formula II with a compound of formula III in the presence of cuprous iodide and Pd(PQ₃)₂Cl₂ or a similar complex. Compounds of formula II and formula III are as follows: 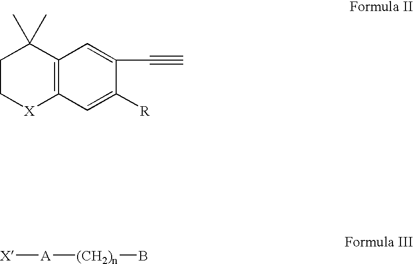
where X═ is a halogen, preferably I; n and A are the same as defined above; and B is H, or a protected acid, alcohol, aldehyde or ketone, giving the corresponding compound of formula I.

Alternately, the compounds of formula I can be made by reacting a zinc salt of formula IV with a compound of formula III in the presence of Pd(PQ₃)₄ (Q is phenyl) or a similar complex, 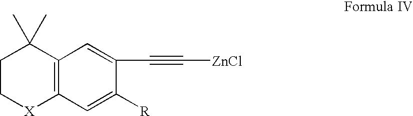
giving the corresponding compound of formula I.

Further, the compounds of formula I can be made by homologating a compound of formula V 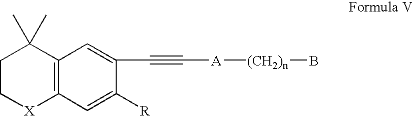
where
n is 0-1 to give an acid of formula I; or
converting an acid of formula I to a salt; or
forming an acid addition salt; or
converting an acid of formula I to an ester; or
converting an acid of formula I to an amide; or
reducing an acid of formula I to an alcohol or aldehyde; or
converting an alcohol of formula I to an ether or ester; or
oxidizing an alcohol of formula I to an aldehyde; or
converting an aldehyde of formula I to an acetal; or
converting a ketone of formula I to a ketal.

The term “ester” as used here refers to and covers any compound falling within the definition of that term as classically used in organic chemistry. Where A is —COOH, this term covers the products derived from treatment of this function with alcohols. Where the ester is derived from compounds where A is —CH₂OH, this term covers compounds of the formula —CH—₂OOCR where R is any substituted or unsubstituted aliphatic, aromatic or aliphatic-aromatic group.

Preferred esters are derived from the saturated aliphatic alcohols or acids of about 10 or fewer carbon atoms or the cyclic or saturated aliphatic cyclic alcohols and acids of about 5 to about 10 carbon atoms. Particularly preferred aliphatic esters are those derived from lower alkyl acids and alcohols. Here, and where ever else used, lower alkyl means having 1 to about 6 carbon atoms. Also preferred are the phenyl or lower alkylphenyl esters.

Amide has the meaning classically accorded that term in organic chemistry. In this instance, it includes the unsubstituted amides and all aliphatic and aromatic mono- and di-substituted amides. Preferred amides are the mono- and di-substituted amides derived from the saturated aliphatic radicals of about 10 or fewer carbon atoms or the cyclic or saturated aliphatic-cyclic radicals of about 5 to about 10 carbon atoms. Particularly preferred amides are those derived from lower alkyl amines. Also preferred are mono- and di-substituted amides derived from the phenyl or lower alkylphenyl amines. Unsubstituted amides are also preferred.

Acetals and ketals include the radicals of the formula —CK where K is (—OR)₂. Here, R is lower alkyl. K may also be —OR₁O— where R₁ is lower alkyl of about 2 to about 5 carbon atoms, straight chain or branched.

A pharmaceutically acceptable salt may be prepared for compounds having a functionality capable of forming such salt, for example, an acid amine functionality. A pharmaceutically acceptable salt may be any salt which retains the activity of the parent compound and does not impart any substantial or significant deleterious or untoward effect on the subject to which it is administered and in the context in which it is administered.

Such a salt may be derived from any organic or inorganic acid or base. The salt may include a mono or polyvalent ion. Of particular interest where the acid function is concerned are the inorganic ions such as sodium, potassium, calcium, magnesium and the like. Organic amine salts may be made with amines, such as mono-, di- and trialkyl amines or alkanol, e.g., ethanol and the like, amines. Salts may also be formed with caffeine, tromethamine and similar molecules. Where there is a nitrogen sufficiently basic as to be capable of forming an acid addition salt, such may be formed with any inorganic or organic acid or alkylating agent, such as methyl iodide. Preferred salt are those formed with inorganic acids such as hydrochloric acid, sulfuric acid, phosphoric acid and the like. Any of a number of simple organic acids, such as a mono-, di- or tri-acid may also be used.

Preferred retinoid components for use in the present invention include those where the ethynyl group and the B group are attached to the 2 and 5 positions respectively of a pyridine ring (the 6 and 3 positions in the nicotinic acid nomenclature being equivalent to the 2/5 designation in the pyridine nomenclature) or the 5 and 2 positions respectively of a thiophene group respectively; n is 0; and B is —COOH, an alkali metal salt or organic amine salt, or a lower alkyl ester, or —CH₂OH and the lower alkyl esters and ethers thereof, or —CHO and acetal derivatives thereof.

More preferred compounds for use in the present invention include:
ethyl 6-(2-(4,4-dimethylthiochroman-6-yl)ethynyl)-nicotinate;
6-(2-4,4-dimethylthiochroman-6-yl)ethynyl) nicotinic acid;
6-(2-4,4-dimethylchroman-6-yl)ethynyl) nicotinic acid;
ethyl 6-2-(4,4-dimethylchroman-6-yl)ethynyl) nicotinate;
ethyl 6-2-(4,4,7-trimethylthiochroman-6-yl)-ethynyl)-nicotinate;
ethyl 6-2-(4,4-dimethyl-1,2,3,4-tetrahydro-quinolin-6-yl)ethynyl)nicotinate;
ethyl 5-2-(4,4-dimethylthiochroman-6-yl) ethynyl)thiophene-2-carboxylate;
6-(2-(4,4-dimethylthiochroman-6-yl)ethynyl)-3-pyridylmethanol; and
2-(2-(4,4-dimethylthiochroman-6-yl)-ethynyl)-5-pyridinecarboxaldehyde.

These compounds, and methods of making these compounds are described in Chandraratna U.S. Pat. No. 5,089,509, the disclosure of which is incorporated in its entirety herein by reference.

A class of useful retinoid components has the structure: 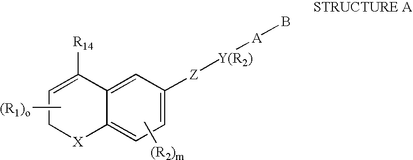
wherein X is S, O, NR′ where R′ is H or alkyl of 1 to 6 carbons, or
X is [C(R₁)₂]ₙ where R₁ is independently H or alkyl of 1 to 6 carbons, and n is an integer between, and including, 0 and 2, and;
R₂ is hydrogen, lower alkyl of 1 to 6 carbons, F, Cl, Br, I, CF₃, fluoro substituted alkyl of 1 to 6 carbons, OH, SH, alkoxy of 1 to 6 carbons, or alkylthio of 1 to 6 carbons, and;
R₃ is hydrogen, lower alkyl of 1 to 6 carbons or F, and;
m is an integer having the value of 0-3, and;
o is an integer having the value of 0-3, and;
   Z is —C≡C—,
   —N═N—,
   —N═CR₁—,
   —CR₁═N,
   —(CR₁═CR₁)_{n′}— where n′ is an integer having the value 0-5,
   —CO—NR₁—,
   —CS—NR₁—,
   —NR₁—CO,
   —NR₁—CS,
   —COO—,
   —OCO—;
   —CSO—;
   —OCS—;
Y is a phenyl or naphthyl group, or heteroaryl selected from a group consisting of pyridyl, thienyl, furyl, pyridazinyl, pyrimidinyl, pyrazinyl, thiazolyl, oxazolyl, imidazolyl and pyrrazolyl, said phenyl and heteroaryl groups being optionally substituted with one or two R₂ groups, or
when Z is —(CR₁═CR₁)_{n′}—and n′ is 3, 4 or 5 then Y represents a direct valence bond between said (CR₂═CR₂)_{n′} group and B;
A is (CH₂)_{q} where q is 0-5, lower branched chain alkyl having 3-6 carbons, cycloalkyl having 3-6 carbons, alkenyl having 2-6 carbons and 1 or 2 double bonds, alkynyl having 2-6 carbons and 1 or 2 triple bonds;
B is hydrogen, COOH or a pharmaceutically acceptable salt thereof, COOR₈, CONR₉R₁₀, —CH₂OH, CH₂OR₁₁, CH₂OCOR₁₁, CHO, CH(OR₁₂)₂, CHOR₁₃O, —COR₇, CR₇(OR₁₂)₂, CR₇OR₁₃O, or tri-lower alkylsilyl, where R₇ is an alkyl, cycloalkyl or alkenyl group containing 1 to 5 carbons, R₈ is an alkyl group of 1 to 10 carbons or trimethylsilylalkyl where the alkyl group has 1 to 10 carbons, or a cycloalkyl group of 5 to 10 carbons, or R₈ is phenyl or lower alkylphenyl, R₉ and R₁₀ independently are hydrogen, an alkyl group of 1 to 10 carbons, or a cycloalkyl group of 5-10 carbons, or phenyl or lower alkylphenyl, R₁₁ is lower alkyl, phenyl or lower alkylphenyl, R₁₂ is lower alkyl, and R₁₃ is divalent alkyl radical of 2-5 carbons, and
R₁₄ is (R₁₅)ᵣ-phenyl, (R₁₅)ᵣ-naphthyl, or (R₁₅)ᵣ heteroaryl where the heteroaryl group has 1 to 3 heteroatoms selected from the group consisting of O, S and N, r is an integer having the values of 0-5, and
R₁₅ is independently H, F, Cl, Br, I, NO₂, N(R₈)₂, N(R₈)COR₈, NR₈CON(R₈)₂, OH, OCOR₈, OR₈, CN, an alkyl group having 1 to 10 carbons, fluoro substituted alkyl group having 1 to 10 carbons, an alkenyl group having 1 to 10 carbons and 1 to 3 double bonds, alkynyl group having 1 to 10 carbons and 1 to 3 triple bonds, or a trialkylsilyl or trialkylsilyloxy group where the alkyl groups independently have 1 to 6 carbons.

Such compounds can be made using well-known techniques. For example, see Klein et al U.S. Pat. No. 5,776,699, the disclosure of which has previously been incorporated in its entirety herein by reference. Therefore, a more detailed express description of the techniques or methods for working such compounds is not presented here.

One particularly useful class of retinoid components for use in the present invention is selected from active acetylenic retinoid agents, precursors of active acetylenic retinoid agents and mixtures thereof. Active acetylenic retinoid agents includes active retinoid agents including at least one —C≡C— group. Examples of such retinoid components are set forth elsewhere herein.

The methods of the present invention are useful in the treatment of nodulocystic acne, for instance, severe nodulocystic acne, and are particularly beneficial because they result in less reduction, or even substantially no reduction, in sebum secretion, for example, which reduction in sebum secretion often occurs with other retinoid agents. Such use of retinoid components in accordance with the present invention effectively provides treatment of nodulocystic acne without subjecting the patient to undue reduction in sebum secretion previously associated with treating nodulocystic acne with other retinoid active agents, for example, isotretinoin.

The present methods provide for less, preferably for substantially no, reduction in sebum secretion.

Especially useful retinoid components useful in the present methods include tazarotene, tazarotenic acid and mixtures thereof. Tazarotene is an ethyl ester prodrug that is metabolized to the corresponding free acid, tazarotenic acid. Tazarotene has a rigid ring-locked structure that offers limited conformational flexibility compared to all-trans-retinoic acid, the natural ligand for the retinoic acid receptors (RARs). This structural change confers tazarotenic acid with specificity for the RARs and selectivity for RAR-β and RAR-γ. As RAR-γ is the major receptor found in skin, tazarotene exerts its pharmacological effects through RAR-γ. Tazarotene is also a potent AP1 antagonist. AP1 regulates the transcription of many genes involved in proliferation and inflammation.

Tazarotenic acid does not activate the RXRs and its major metabolite, the sulfoxide AGN 190844, does not activate either the RARs or the RXRs. As it has no isomerizable double bonds, tazarotene cannot be converted into RXR-active compounds. In contrast, polyolefinic retinoids such as isoretinoin and acitretin can be isomerized and the isomers could potentially activate the RARs and/or RXRs. RXR agonists cause transient elevation of triglycerides by inhibiting peripheral tissue lipoprotein lipase activity. RAR and RXR ligands act synergistically to induce hypertriglyceridemia. RAR pan agonists also induce hypertriglyceridemia by increasing hepatic triglyceride output, and this effect is primarily mediated by the RAR-A receptor. RAR-γ is not implicated in hypertriglyceridemia. As tazarotenic acid has minimal RAR-A activity and substantially no RXR activity, it would not be expected to significantly elevate triglycerides—by either of the pathways.

Clinical use of RXR agonists has also been associated with hypothyroidism. As tazarotene is RAR specific, and cannot be either metabolized or isomerized to RXR active compounds, it would not be expected to cause either significant elevation of triglycerides or hypothyroidism.

The substantial absence of RXR activity and the minimal RAR-α activity of tazarotenic acid are important factors that reduce the potential for some toxicities, such as hypertriglyceridemia and hypothyroidism, that are typically associated with oral retinoids.

The LC-MS/MS test for simultaneous detection of tazarotene and tazarotenic acid may be run as follows. One ml of plasma (EDTA-treated) is diluted with 1.0 ml of water. Diluted plasma is extracted using solid phase extraction (SPE) on a C18 cartridge. The eluate is evaporated, reconstituted in a water/methanol-based mobile phase, and injected onto a 4.6×50 mm, 3 μm pore size C-8 reverse phase high pressure liquid chromatography (HPLC) column (Agilent, Wilmington, Del.). Compounds are gradient-eluted at 1.2 mL/min and detected using an API 3000 triple quadrupole mass spectrometer with an Atmospheric Pressure Chemical Ionization (APCI) source (PE-Sciex, Concord, Ontario, Canada). Molecular reaction monitoring enhances the sensitivity and selectivity of this assay by collisionally dissociating the protonated molecules for the analyte and an internal standard thereby forming the product ions. The specific precursor-product ion pair monitored are m/z 352→324 for tazarotene, m/z 359→331 for the tazarotene internal standard, m/z 324→294 for tazarotenic acid, and m/z 331+298 for the tazarotenic acid internal standard. The lower limit of quantitation at assay range tested is 0.1 ng/mL, with a coefficient of variation and deviation from nominal concentration of <15%.

Retinoid components useful in the present invention may be included in a composition with one or more other suitable pharmaceutically acceptable ingredients. Examples of useful other ingredients include, but are not limited to antioxidants, such as butylated hydroxyanisole NF and the like; emulsifiers, such as sorbitan monoolate NF, polysorbate 80 NF and the like and mixtures thereof; vehicle components, such as conventional vehicles and the like; and other materials which are useful to provide one or more benefits to the composition to be administered and/or to the subject to whom the composition is administered.

Daily dosages of the presently useful retinoid components may vary from patient to patient depending, for example, on the desired therapeutic effect to be achieved, on the condition of the patient, on the mode of systemic administration, on the frequency of administration and the like factors. Such dosages advantageously are selected to provide the desired therapeutic effect, preferably substantially without unduly harming or interfering with the patient. Examples, without limitation, of such daily dosages may be in a range of about 0.1 mg/day or less or about 0.3 mg/day to about 7 mg/day or about 10 mg/day or more.

When the desired therapeutic effect is a reduction in nodulocystic acne, for example, severe nodulocystic acne, daily dosages are often within the above-noted ranges. When tazarotene is orally administered to effect a reduction in such acne, the daily dosage of tazarotene preferably is in a range of about 0.3 mg/day to about 7 mg/day or about 8 mg/day, more preferably in a range of about 0.6 mg/day to about 6.5 mg/day or about 7 mg/day. Clinical trials using orally administered tazarotene to effect reductions, as described elsewhere herein, in nodulocystic acne have employed daily dosages of tazarotene including 0.4 mg/day, 0.75 mg/day, 1.5 mg/day, 2.8 mg/day, 3 mg/day, 4.5 mg/day, 6 mg/day and 6.3 mg/day.

Although the presently useful retinoid components can be advantageously administered on a once daily basis, other dosing frequencies may be employed. For example, the presently useful retinoid components may be administered twice or three or more times daily, or once every two or three or more days.

The following non-limiting examples illustrate certain aspects of the present invention.

### EXAMPLE 1

Coadministration of 6.3 mg oral tazarotene with a high-fat meal in normal healthy subjects following single and multiple dose administrations does not substantially affect the bioavailability or pharmacokinetics of tazarotenic acid, the primary active retinoid species in the systemic circulation. This result is based on comparing the pharmacokinetics of tazarotenic acid when administered within 30 minutes after consuming a high fat breakfast vs. when administered after an 8-10 hour fast. The 90% confidence intervals (CI) of AUC ratios (test/reference) are completely within the 80-125% boundary. The 90% CI ratio of Cₘₐₓ values are partially outside the 80-125% boundary due to data variability, but the average ratios of 1.00 (Day 0) and 0.829 (Day 9) are within the above-noted limit.

Each of isotretinoin, acitretin and bexarotene is commercially available as an oral active retinoid agent. To one degree or another, patients taking each of these agents are instructed to take the agent with food to achieve improved drug absorption or bioavailability. This substantial dependence on the presence of food to achieve improved bioavailability clearly distinguishes these agents from the retinoid components useful in the present invention.

### EXAMPLE 2

A series of Phase 3 studies are conducted on orally administered tazarotene for the treatment of psoriasis. Adverse events (side effects) are monitored. Results of such monitoring are shown in Table 1. In addition, published data for acitretin's side effects are also considered. Table 1 shows the adverse events reported for at least 10% of the patients in the studies of acitretin versus those seen with tazarotene.

TABLE 1

| | NUMBER OF (%) OF PATIENTS | | | |
|---|---|---|---|---|
| | | Taz 4.5 mg | Taz 4.5 mg | |
| | | Study 3 | Study 3 | |
| | Taz 4.5 mg | treated | treated | |
| | Combined | with | with | |
| | data from | Tazarotene | Tazarotene | |
| | Studies 1 | for 6 | for 3 | |
| | and 2 | months | months | Acitretin |
| Adverse Event | (N = 348) | (N = 92) | (N = 220) | (N = 525) |
| Cheilitis | 228 (65.5) | 65 (70.7) | 149 (67.7) | 429 (81.7) |
| Skin | 3 (0.9) | 1 (1.1) | 3 (1.4) | 345 (65.7) |
| peeling/ | | | | |
| Desquamation | | | | |
| Alopecia | 1 (0.3) | 5 (5.4) | 1 (0.5) | 319 (60.8) |
| Dry Skin | 82 (23.6) | 24 (26.1) | 47 (21.4) | 174 (33.1) |
| Nail Disorder | 2 (0.6) | 1 (1.1) | 1 (0.5) | 170 (32.4) |
| Pruritus | 21 (6.0) | 3 (3.3) | 11 (5.0) | 157 (29.9) |
| Rhinitis | 8 (2.3) | 0 (0.0) | 0 (0.0) | 153 (29.1) |
| Sticky skin/Skin | 1 (0.3) | 0 (0.0) | 0 (0.0) | 129 (24.6) |
| disorder | | | | |
| Xerophthalmia/ | 8 (2.3) | 1 (1.1) | 3 (1.4) | 112 (21.3) |
| Eye disorder | | | | |
| Arthralgia | 60 (17.2) | 30 (32.6) | 28 (12.7) | 102 (19.4) |
| Dry mouth/Oral | 11 (3.2) | 2 (2.2) | 11 (5.0) | 76 (14.5) |
| dryness | | | | |
| Rigors/Chills | 9 (2.6) | 1 (1.1) | 1 (0.5) | 67 (12.8) |
| Hyperesthesia | 0 (0.0) | 0 (0.0) | 0 (0.0) | 66 (12.6) |
| Paronychia/Nail | 2 (0.6) | 1 (1.1) | 1 (0.5) | 66 (12.6) |
| disorder | | | | |
| Atrophy of skin | 0 (0.0) | 0 (0.0) | 0 (0.0) | 64 (12.2) |
| Epistaxis | 2 (0.6) | 0 (0.0) | 1 (0.5) | 61 (11.6) |
| Erythematous | 2 (0.6) | 0 (0.0) | 0 (0.0) | 57 (10.9) |
| rash/ | | | | |
| Erythema | | | | |
| Paresthesia | 7 (2.0) | 1 (1.1) | 3 (1.4) | 56 (10.7) |
| Back pain | 16 (4.6) | 10 (10.9) | 7 (3.2) | 41 (7.8) |

The results shown in Table 1 demonstrate that tazarotene (in a therapeutically effective amount) when used to treat psoriasis in accordance with the present invention results in substantial reductions in, and even in some cases elimination of, side effects relative to the side effects resulting from the administration of acitretin.

### EXAMPLE 3

Two multicenter, double-blind, randomized, placebo-controlled 24-week studies of identical design are conducted to evaluate the safety of oral tazarotene. In addition, 16- to 24-week dose-response evaluations are performed.

In the two safety trials, the incidence of adverse side effects with a 4.5 mg dose administered orally once daily is compared to the incidence of the same side effects with a placebo. The following side effects are found to occur significantly more frequently with tazarotene than with the placebo: cheilitis (66% vs 17%), dry skin (24% vs 15%), headache (19% vs 12%), arthralgia (17% VS 8%), myalgia (14% vs 8%), back pain (7% vs 3%), joint disorder (4% vs 1%), nasal dryness (4% vs 1%), foot pain (3% vs 1%), rash (3% vs 1%), hyperglycemia (2% vs 0%), and dermatitis (1% vs 0%). The majority of these were mild in severity and typical of adverse effects associated with oral retinoids. Particularly noteworthy is that other adverse effects typically associated with oral retinoids—including hypertriglyceridemia, hypercholesterolemia, abnormal liver function tests, increased alanine aminotransferase, increased aspartate aminotransferase, desquamation, eye dryness, and alopecia—occur with essentially the same incidence with oral tazarotene as with placebo.

There are no statistically significant between-group difference in the incidence of ocular, auditory, or thyroid problems. Altered hormone levels (which includes elevated TSH and T4, decreased T4, and abnormal thyroid function test) occur significantly more frequently with placebo (2%) than with tazarotene (0%).

There is also no evidence that tazarotene was associated with an increased incidence of psychiatric disorders—depression (1% with tazarotene vs 2% with placebo), psychosis (0% vs <1%), psychotic depression (0% vs <1%), emotional lability (3% vs 3%), anxiety (1% vs <1%), and agitation (<1% vs 0%).

Data from the two identical trials plus the dose-response evaluations show that the incidence of patients discontinuing due to adverse effects is approximately 2% with 0.4 to 1.1 mg oral tazarotene (n=105), 10% with 2.1 to 2.8 mg oral tazarotene (n=21), 0% with w.2 mg (n=14), 3% with 4.5 mg (n=348), 13% with 6.3 mg (n=16), and 3% with placebo (n=383).

The results show that oral tazarotene appears to have safety and tolerability advantages over many other systemic treatments.

### EXAMPLE 4

Two multicenter, double-blind, placebo-controlled studies ware conducted to evaluate the efficacy and safety of oral tazarotene (4.5 mg once daily) in patients with moderate to very severe plaque psoriasis. Patients who did not respond to 12 weeks of treatment with oral tazarotene (n=89) or placebo (n=217) in those studies are eligible to enter an open-label extension study in which they receive oral tazarotene (4.5 mg once daily) for 12 weeks and are then followed without treatment for an additional 12 weeks. Efficacy evaluations include overall lesional assessment (OLA), percent body surface area involvement, global response to treatment, plaque elevation, scaling, and erythema. OLA is graded on a 6-point scale (0=none, 1=minimal, 2=mild, 3=moderate, 4=severe, 5=very severe).

Except in the initial few weeks of treatment, there are no statistically significant differences for any efficacy variable between the group previously treated with tazarotene and the group previously treated with placebo. Clinical success (a 2-grade reduction in OLA, the primary efficacy variable) is attained in 28% of the patients at the end of the 12-week treatment period. At the end of the post-treatment period, 16% of the patients have clinical success.

The overall incidence of adverse events during the treatment period of the extension study is not significantly different in the group previously treated with tazarotene compared with the group previously treated with placebo. Overall, (i.e., across both groups) the most commonly reported adverse events are cheilitis (incidence of 70%), dry skin (25%), arthralgia (20%), myalgia (15%), headache (11%), back pain (11%), infection (10%), hypertriglyceridemia (6%), and asthenia (6%). The majority of the events are mild in severity. There are no serious adverse events considered related to the study treatment and no clinically significant changes in liver function test values or cholesterol levels.

The results show that continued treatment with oral tazarotene can offer good efficacy in patient initially unresponsive to oral tazarotene or placebo treatment. Oral tazarotene has a good safety profile and is well tolerated, with the majority of adverse events being mild.

### EXAMPLE 5

Two placebo-controlled dose-ranging studies are conducted to evaluate the safety of oral tazarotene in patients having nodulocystic acne. In the first study, patients receive orally administered tazarotene at daily doses of 0.4 mg to 2.8 mg in 96 (71+25) patients (12 weeks treatment plus 12 weeks post-treatment). In the second study, daily doses of 0.75 mg to 6 mg tazarotene are administered to 181 patients (24 weeks treatment plus 12 weeks post-treatment).

Oral tazarotene is well tolerated, with only 2.5% (7/277) of patients withdrawing from either study due to adverse events (2 each with placebo, 0.75 mg, and 3 mg, and 1 with 6 mg).

The most common adverse events occurring during the treatment period in the placebo groups combined (n=54) or the three highest tazarotene dose groups (2.8 mg, n=11; 3 mg, n=37; and 6 mg, n=36) include cheilitis (31%, 64%, 78%, and 94%, respectively), dry skin (19%, 18%, 35%, 50%), headache (28%, 9%, 19%, 36%), arthralgia (6%, 9%, 8%, 28%), myalgia (9%, 0%, 16%, 25%), joint disorder (0%, 27%, 14%, 19%), and asthenia (13%, 0%, 19%, 19%). These events are predominantly mild or moderate in severity. For example, in the 3 mg and 6 mg groups, cheilitis is mild in 25 and 27 patients, respectively, moderate in 3 and 6 patients, and severe in 1 patient in each group. In the same groups, dry skin is mild in 11 and 17 patients, respectively, and moderate in 2 and 1 patient. No patient has severe dry skin.

Emotional lability occurs in 1 (3%) patient in the 3 mg group and 5 (14%) in the 6 mg group compared with 2 (4%) with placebo. All cases of emotional lability are mild. Depression occurs in 3 (8%) patients in the 3 mg group (2 mild, 1 severe), none in the 6 mg group, and 1 (2%)(moderate) with placebo.

There are no consistent dose-related clinically significant changes in urinalysis, chemistry, or hematology measures (including the results of liver function tests and levels of triglycerides, total cholesterol, and HDL cholesterol). Tazarotene treatment is also not associated with any clinically significant ligament calcification, osteophyte formation, or changes in serum bone alkaline phosphatase, serum amino terminal telopeptides, or bone density.

The results suggest that oral tazarotene has a good safety and tolerability profile in the treatment of nodulocystic acne and does not appear to result in clinically significant changes in liver enzymes, cholesterol or triglyceride levels, or bone density.

### EXAMPLE 6

Studies are conducted on orally administered tazarotene for the treatment of acne. Adverse events (side effects) are monitored.

Results of such monitoring are shown in Table 2. Published data for isotretinoin's side effects are also considered. Table 2 shows the adverse events reported for at least 10% of the patients in the studies of isotretinoin versus those for tazarotene.

TABLE 2

NUMBER OF (%) OF PATIENTS

| | Tazarotene | |
|---|---|---|
| | (N = 223) | |
| | Combined data | |
| | from Studies 1 | Isotretinoin |
| Adverse Event | and 2 | (N = 525) |
| Cheilitis | 134 (60.1) | 472 (90.2) |
| Conjunctivitis | 0 (0.0) | 201 (38.4) |
| Desquamation central | 1 (0.4) | 187 (35.8) |
| face/Desquamation | | |
| Dry skin | 79 (35.4) | 172 (32.9) |
| Skin fragility/Skin | 1 (0.4) | 164 (31.4) |
| disorder | | |
| Dry nose | 7 (3.1) | 136 (26.0) |
| Pruritus | 10 (4.5) | 127 (24.3) |
| Epistaxis | 6 (2.7) | 121 (23.1) |
| Peeling/Desquamation | 1 (0.4) | 109 (20.8) |
| Irritation of eyes | 0 (0.0) | 85 (16.3) |
| Rash-dermatitis/Rash | 7 (3.1) | 82 (15.7) |
| Fingertip | 1 (0.4) | 63 (12.0) |
| peeling/Desquamation | | |
| Joint pain/Arthralgia | 18 (8.1) | 60 (11.5) |
| Red Scaly | 1 (0.4) | 54 (10.3) |
| face/Desquamation | | |
| Headache | 26 (11.7) | 44 (8.4) |

The results shown in Table 2 demonstrate that tazarotene (in a therapeutically effective amount) when used to treat acne in accordance with the present invention results in substantial reductions in, and even in some cases elimination of, side effects relative to the side effects resulting from the administration of isotretinoin.

### EXAMPLE 7

A clinical study of healthy human volunteers having differing body weights involving the oral administration of tazarotene is conducted. Each of the subjects is administered a single daily dose of 6 mg of tazarotene. The plasma of each subject is tested for Cₘₐₓ and AUC of tazarotenic acid, the primary active retinoid agent in systemic circulation as the result of the oral administration of tazarotene. Comparisons of plasma tazarotenic acid Cₘₐₓ and AUC values among subjects of differing body weights show no effect of body weight on systemic tazarotenic acid exposure (p>0.05). Specifically, while there may be a correlation of AUC with body weight on day 0, there is no such correlation with Cₘₐₓ, and there is no correlation between either Cₘₐₓ or AUC and body weight on day 9.

Each of isotretinoin, acitretin and bexarotene is commercially available as an oral active retinoid agent. To one degree or another, these agents are prescribed for and administered to a patient based on the body weight of the patient in order to achieve improved drug bioavailability. This substantial dependence on body weight to achieve improved bioavailability clearly distinguishes these agents from the retinoid components useful in the present invention.

Each of isotretinoin, acitretin and bexarotene is commercially available as an oral active retinoid agent. To one degree or another, patients taking each of these agents are instructed to take the agent with food to achieve improved drug absorption or bioavailability. This substantial dependence on the presence of food to achieve improved bioavailability clearly distinguishes these agents from the retinoid components useful in the present invention.

### EXAMPLE 8

All oral retinoids require female patients of childbearing potential to use reliable birth control measures during treatment and for varying periods of time after treatment.

It is the objective of this study to determine if there are pharmacokinetic (PK) and pharmacodynamic (PD) interactions between tazarotene and commonly prescribed oral contraceptives (OCs) when coadministered together.

Three separate clinical studies are conducted to evaluate the PK and PD interactions of oral tazarotene and OCs in healthy volunteers. Two studies evaluate daily doses of norethindrone (NE)/ethinyl estradiol (EE) with daily doses of tazarotene 1.1 mg (N=27) and 6 mg (N=29). The third study assesses the daily doses of norgestimate (NGM)/EE with daily doses of tazarotene 6 mg (N=26). OCs are administered for three consecutive menstrual cycles. Daily doses of tazarotene are started during the 2nd cycle and continued through the end of the studies. PK parameters for EE, NE, and NGM (AUC₀₋₂₄ and Cₘₐₓ) are determined before tazarotene dosing and after tazarotene dosing on day 6 of the 2nd and 3rd cycles. Serum concentrations of luteinizing hormone (LH) and follicle-stimulating hormone (FSH)-markers of contraceptive efficacy are also evaluated before tazarotene dosing and after tazarotene dosing on days 2, 4, and 6 of the 2nd and 3rd cycles. Serum progesterone levels are assessed on days 18 and 20 of the 2nd and 3rd cycles.

The plasma of each subject is tested for Cₘₐₓ and AUC of tazarotenic acid, the primary active retinoid agent in systemic circulation as the result of the oral administration of tazarotene.

Results

The 90% confidence intervals of AUC₀₋₂₄ and Cₘₐₓ for EE, NE, and NGM in each of the studies are within the 80-125% boundary, indicating tazarotenic acid, the active retinoid agent resulting from the oral administration of tazarotene, does not affect the pharmacokinetics of the components of the two OCs. Similarly, the 90% confidence intervals of progesterone concentrations are within the 80-125% boundary. The 90% confidence intervals of FSH and LH are generally within the 80-125% boundary with some scatter due to data variability.

The mean concentrations of FSH and LH are lower in the 3rd cycle than the 2nd cycle on some days, indicating that the efficacy of the OCs is not compromised by the tazarotene administration. The serum FSH and LH levels remain within the normal ranges for healthy women during the follicular phase.

These data demonstrate that orally administered tazarotene, up to 6 mg once daily, does not affect the PK or efficacy of NE/EE and NGM/EE oral contraceptives.

### EXAMPLE 9

The patient, a woman 26 years of age, presents symptoms of severe psoriasis. The symptoms include lesions approximately 4 to 10 centimeters across appearing as raised patches of wine red skin many of which are covered in silvery white scales. The lesions are mostly dry and rough, and quite often noticeably warm to the touch. The lesions are present on the elbows, knees, scalp, and groin area. The patient experiences intense burning and itching associated with the lesions.

The patient is currently taking Ortho-Novum® for contraception.

Tazarotene at a dose of 6 mg per day is prescribed. After 30 days of administration, the patient's symptoms of itching and burning are relieved and the severity of her lesions are substantially lessened.

During the course of administration, the patient's plasma concentrations of FSH and LH are lower in the 3rd cycle than the 2nd cycle on some days, indicating that the efficacy of the oral contraceptive is not compromised by the tazarotene administration. The serum FSH and LH levels remain within the normal ranges for a healthy women during the follicular phase.

The observed AUC₀₋₂₄ and Cₘₐₓ values for tazarotenic acid during the period of administration are 379±78 ng·hr/ml and 111±37 ng/mL (mean±SD), respectively.

### EXAMPLE 10

A multicenter, double-blind, randomized, placebo-controlled parallel-group study is undertaken to determine the efficacy of orally administered tazarotene in treating severe nodulocystic acne.

The main inclusion criteria for this study include: at least 7 facial nodulocystic acne lesions (>5 mm); an age of at least 16 years; stable doses of any concurrent medication that might significantly affect hepatic or renal excretion; if taking oral contraceptives, stable dose for last 3 months; and negative urine pregnancy test for females of childbearing potential.

The main exclusion criteria for this study include: females of childbearing potential not committed to using highly effective contraceptive during the study; pregnant or lactating females; 8-hour fasting triglyceride levels ≧500 mg/dL, serum calcium levels >11 mg/dL; likelihood of prolonged exposure to ultraviolet light during the study; and uncontrolled systemic disease.

In addition, washout periods for other medications for this study are: 1 week for vitamin A supplements >5000 IU; 2 weeks for topical anti-acne medications (e.g., retinoids, azelaic acid, benzoyl peroxide); 2 weeks for topical or systemic antibiotic therapy that may alter the course of acne; and 6 months for systemic retinoids.

Treatment Regimen

Patients are randomized to receive placebo or oral tazarotene (0.75, 1.5, 3, or 6 mg), in a 1:1:1:1:1 ratio once daily for 24 weeks. After this, the patients are followed without treatment for an additional 12 weeks. Patients discontinuing from the treatment period due to adverse effects or lack of efficacy are eligible for entry into the post-treatment phase.

Main Outcome Measures

In the study, overall acne severity is rated as: none (no inflammatory acne lesions); mild (few to several papules or pustules, no nodulocystic lesions); moderate (several to many papules or pustules, few to several nodulocystic lesions); and severe (numerous or extensive papules or pustules, many, for example, at least about 5 or at least about 10, nodulocystic lesions).

Treatment success is defined as at least moderate (about 50%) improvement on the following 7-point global response scale:
0=completely cleared
1=almost cleared (about 90% improvement)
2=marked response (about 75% improvement)
3=moderate response (about 50% improvement)
4=slight response (about 25% improvement)
5=condition unchanged
6=condition worsened

In this study, facial nodulocystic lesion count includes lesions greater than 5 mm in size. Facial papular/pustular lesion count includes lesions less than or equal to 5 mm in size. Facial non-inflammatory lesion count includes open and closed comedones.

Other Measures

Sebum output is assessed every 4 weeks at selected centers using the Sebutape® patches, sold by Cuderm Corporation. Urinalysis, chemistry, and hematology values are monitored. Bone formation and resorption assessments (serum bone alkaline phosphatase and serum amino terminal telopeptides, respectively) at selected centers are monitored.

Bone mineral density of spine and proximal femur at selected centers is monitored. Ligament calcification or osteophyte formation (lateral X-ray of the cervical and thoracic spine, and ankle calcaneous) is monitored. Epiphyseal growth plate closure (internal oblique X-ray of the ankle in patients less than or equal to 21 years old) is monitored.

Results

Patients

181 patients enroll in the study. 127 (70%) of the patients complete the 24-week treatment phase. 145 patients enter the 12-week post-treatment phase. 96 (66%) of these patients complete this phase. The study population is nearly equally divided between males and females (55% males) and is ethnically diverse (61% Caucasian, 22% Hispanic, 12% black, 4% Asian, 1% other). The mean age is 22.7 years. The mean number of facial nodulocystic lesions at baseline ranges from 10.8 to 12.2 in the treatment groups. There are no significant differences between the groups at baseline in demographics or measures of acne severity.

In the treatment period, few patients withdraw due to adverse events that are unrelated to or possibly or definitely related to treatment. The withdrawing patients are as follows: 0% (0/36) of placebo group; 6% (2/35) of 0.75 mg group (anxiety attack, mild leucopenia); 0% (0/37) of 1.5 mg group; 5% (2/37) of 3 mg group (infectious mononucleosis, depression); and 3% (1/36) of 6 mg group (spinal stiffness and joint and muscle pain).

Patients withdrawing due to lack of efficacy are primarily in the placebo or lowest dose groups: 17% (6/36) of placebo group; 20% (7/35) of 0.75 mg group; 5% (2/37) of 1.5 mg group; 0% (0/37) of 3 mg group; and 6% (2/36) of 6 mg group.

Main Outcome Measures

Results of this study are that tazarotene at 6 mg reduces the overall acne severity significantly more than placebo from week 16 until the end of the post-treatment phase (p≦0.01). More than 45% of the patients in the three highest dose groups have either no acne or mild acne by the end of the treatment phase, compared with 34% in the tazarotene 0.75 mg group and 19% in the placebo group. At the end of the post-treatment phase, these levels of acne have been maintained in 53% of the 6 mg group and 43% of the 3 mg group.

Tazarotene at 3 mg and 6 mg have a significantly higher incidence of treatment success (≧50% global improvement) than placebo at week 24 and throughout the post-treatment phase (p≦0.05). Treatment success is achieved by week 12 in more than 70% of patients treated with the three highest doses of tazarotene and by week 24 in more than 86% of patients treated with the two highest doses. Tazarotene achieves consistently greater reductions in the number of total facial nodulocystic lesions than placebo from week 8 onward. At the end of the treatment period, the mean total facial nodulocystic lesion count is reduced from:
11.6 to 5.2 in the placebo group (a 55% reduction);
12.2 to 4.2 in the 0.75 mg group (a 66% reduction);
11.8 to 3.2 in the 1.5 mg group (a 73% reduction);
10.8 to 2.3 in the 3 mg group (a 79% reduction); and
11.6 to 1.6 in the 6 mg group (an 86% reduction).

The percentage of patients with at least a 90% reduction in facial nodulocystic lesion count is significantly greater in the higher-dose tazarotene groups (1.5, 3, and 6 mg) than in the placebo group at week 24. The 6 mg group also shows significant superiority over placebo at the end of the post-treatment phase. The median time to initial complete clearing of facial nodulocystic lesions is less in the 3 mg and 6 mg groups (16 weeks in both groups) than in the placebo group (24 weeks) (p=0.017 and p=0.081, respectively).

From week 8 onward, tazarotene results in consistently greater reductions in the number of facial papules or pustules, and facial non-inflammatory acne lesions, compared with placebo. At the end of the treatment period, the mean facial papule or pastule count is reduced from:
32.4 to 22.3 in the placebo group (a 31% reduction);
32.3 to 20.3 in the 0.75 mg group (a 37% reduction);
29.1 to 13.3 in the 1.5 mg group (a 54% reduction);
25.4 to 10.0 in the 3 mg group (a 61% reduction); and
24.6 to 11.1 in the 6 mg group (a 55% reduction).

At the end of the treatment period, the mean facial non-inflammatory lesion count is reduced from:
62.3 to 34.2 in the placebo group (a 45% reduction);
56.3 to 30.3 to 30.3 in the 0.75 mg group (a 46% reduction);
59.2 to 17.8 in the 1.5 mg group (a 70% reduction);
56.1 to 21.3 in the 3 mg group (a 62% reduction);
and 47.7 to 13.5 in the 6 mg group (a 72% reduction).

Sebum secretion output is assessed in a maximum of 86 patients (with successively fewer patients at each timepoint—e.g., 60 patients at week 24, 46 patients at week 36). Importantly, there is no consistent statistically significant differences in sebum production across treatment groups using the Sebutape® method of assessment. In other words, the oral administration of tazarotene does not substantially reduce sebum secretion relative to placebo, even when such tazarotene administration is effective to reduce or even eliminate severe nodulocystic acne.

The most common adverse events (i.e., with an incidence of ≧15%) occurring in the treatment period are cheilitis, dry skin, arthralgia, and joint disorder. For each of these adverse events, the majority of cases are mild.

None of the treatment groups show any consistent clinically significant changes in urinalysis, chemistry, or hematology measures (including liver function test results and levels of triglycerides, total cholesterol, and high-density lipoprotein cholesterol).

Bone formation and resorption do not appear to be altered. There are no statistically significant differences between groups in the mean change from baseline in serum bone alkaline phosphatase (bone formation) or serum amino terminal telopeptides (bone resorption).

None of the treatment groups show any clinically significant ligament calcification, osteophyte formation, or changes in bone density. All patients have distal tibial growth plate closure at both day 0 and the last follow-up visit. Two patients have physes that are partially closed at baseline and both close completely during the study in an unremarkable fashion.

The results of this study suggest that oral tazarotene has a better tolerability profile than other oral retinoids. Oral isotretinoin has been associated with several adverse events that did not occur as frequently—or at all—with oral tazarotene. For example, the only adverse effects reported with an incidence of ≧15% with oral tazarotene are cheilitis, dry skin, headache, arthralgia, myalgia, infection, asthenia, and joint disorder. Furthermore, oral tazarotene is not generally associated with abnormalities in liver function test results or elevated levels of triglycerides, total cholesterol, or high-density lipoprotein cholesterol.

Oral tazarotene is efficacious at once-daily doses of 1.5, 3, and 6 mg. The higher doses are associated with the greatest efficacy, the most rapid clearing of facial nodulocystic lesions, and maintenance of response for at least 12 weeks post-treatment. The superiority of the 6-mg dose of oral tazarotene over placebo is significant from week 16 onward. As noted above, oral administration of tazarotene does not substantially reduce sebum secretion, even when such administration is effective to reduce or even eliminate severe nodulocystic acne.

While this invention has been described with respect to various specific examples and embodiments, it is to be understood that the invention is not limited thereto and that it can be variously practiced within the scope of the following claims.

1. A method of treating nodulocystic acne in a human or animal comprising:
orally administering to a human or animal having nodulocystic acne a therapeutically effective amount of a retinoid component selected from the group consisting of tazarotene, tazarotenic acid, derivatives of tazarotene, other precursors of tazarotenic acid, derivatives of tazarotenic acid and mixtures thereof, the administering being effective to provide at least about 60% reduction in nodulocystic acne in the human or animal, and to provide less reduction in sebum secretion in the human or animal relative to employing a reference retinoid agent in place of the retinoid component in an orally administering step using an amount of the reference retinoid agent to provide the same reduction in nodulocystic acne.

2. The method of claim 1 wherein the administering is effective to provide at least about 85% reduction in nodulocystic acne in the human or animal.

3. The method of claim 1 wherein the administering step results in substantially no reduction in sebum secretion in the human or animal.

4. The method of claim 1 wherein the reference retinoid agent is a pan active RAR retinoid agent or an active retinoid agent effective to bind to RXRs.

5. The method of claim 1 wherein the administering step is effective to provide a maximum blood concentration of active retinoid agent in the human or animal of greater than 30 ng/ml.

6. The method of claim 1 wherein the administering step is effective to provide a maximum blood concentration of active retinoid agent in the human or animal of greater than about 45 ng/ml.

7. The method of claim 1 wherein the administering step is effective to provide a maximum blood concentration of active retinoid agent in the human or animal of greater than about 100 ng/ml.

8. The method of claim 1 wherein the administering step comprises orally administering a daily dose in a range of about 1 mg to about 6 mg of the retinoid component to the human or animal.

9. The method of claim 1 wherein the administering step comprises orally administering a capsule containing the retinoid component to the human or animal.

10. The method of claim 1 wherein the administering step comprises repeatedly orally administering the retinoid component to the human or animal for a period of time in excess of about 1 week.

11. The method of claim 1 wherein the administering step comprises repeatedly orally administering the retinoid component to the human or animal for a period of time in excess of about 20 weeks.

12. A method of treating nodulocystic acne in a human or animal comprising:
orally administering to a human or animal having nodulocystic acne a daily dose in a range of about 1 mg to about 6 mg of a retinoid component selected from the group consisting of active retinoid agents, precursors of active retinoid agents and mixtures thereof, the administering being effective to provide at least 60% reduction in nodulocystic acne in the human or animal, and to provide less reduction in sebum secretion in the human or animal relative to employing a reference retinoid agent in place of the retinoid component in an orally administering step using an amount of the reference retinoid agent to provide the same reduction in nodulocystic acne.

13. The method of claim 12 wherein the administering is effective to provide at least about 85% reduction in nodulocystic acne in the human or animal.

14. The method of claim 12 wherein the administering step results in substantially no reduction in sebum secretion in the human or animal.

15. The method of claim 12 wherein the reference retinoid agent is a pan active RAR retinoid agent or an active retinoid agent effective to bind to RXRS.

16. The method of claim 12 wherein the administering step is effective to provide a maximum blood concentration of active retinoid agent in the human or animal of greater than 30 ng/ml.

17. The method of claim 12 wherein the administering step is effective to provide a maximum blood concentration of active retinoid agent in the human or animal of greater than 45 ng/ml.

18. The method of claim 12 wherein the administering step is effective to provide a maximum blood concentration of active retinoid agent in the human or animal of greater than about 100 ng/ml.

19. The method of claim 12 wherein the administering step comprises orally administering a capsule containing the retinoid component to the human or animal.

20. The method of claim 12 wherein the administering step comprises repeatedly orally administering the retinoid component to the human or animal for a period of time in excess of about 1 week.

21. The method of claim 12 wherein the administering step comprises repeatedly orally administering the retinoid component to the human or animal for a period of time in excess of about 20 weeks.

22. The method of claim 12 wherein the retinoid component includes an active retinoid agent or a precursor of an active retinoid agent effective to more selectively affect at least one of RAR-beta and RAR-gamma relative to RAR-alpha.

23. The method of claim 12 wherein the retinoid component includes an active retinoid agent more water soluble than isotretinoin or is converted in the human or animal into an active retinoid agent more water soluble than isotretinoin.

24. The method of claim 12 wherein the retinoid component is substantially ineffective to bind with RXRs.

25. The method of claim 12 wherein the retinoid component is selected from the group consisting of active acetylenic retinoid agents, precursors of active acetylenic retinoid agents and mixtures thereof.

26. The method of claim 12 wherein the retinoid component is selected from the group consisting of tazarotene, tazarotenic acid and mixtures thereof.

27. The method of claim 12 wherein the retinoid component includes tazarotene.

28. A method of treating nodulocystic acne in a human or animal comprising:
orally administering to a human or animal having nodulocystic acne for a period of time in excess of about 1 week a therapeutically effective amount of a retinoid component selected from the group consisting of active retinoid agents, precursors of active retinoid agents and mixtures thereof, the administering being effective to provide at least about 60% reduction in nodulocystic acne in the human or animal, and to provide less reduction in sebum secretion in the human or animal relative to employing a reference retinoid agent in place of the retinoid component in an orally administering step using an amount of the reference retinoid agent to provide the same reduction in nodulocystic acne.

29. The method of claim 28 wherein the administering is effective to provide at least about 70% reduction in nodulocystic acne in the human or animal.

30. The method of claim 28 wherein the administering is effective to provide at least about 85% reduction in nodulocystic acne in the human or animal.

31. The method of claim 28 wherein the administering step results in substantially no reduction in sebum secretion in the human or animal.

32. The method of claim 28 wherein the reference retinoid agent is a pan active RAR retinoid agent or an active retinoid agent effective to bind to RXRS.

33. The method of claim 28 wherein the systemically administering step is effective to provide a maximum blood concentration of active retinoid agent in the human or animal of greater than 30 ng/ml.

34. The method of claim 28 wherein the systemically administering step is effective to provide a maximum blood concentration of active retinoid agent in the human or animal of greater than about 45 ng/ml.

35. The method of claim 28 wherein the systemically administering step is effective to provide a maximum blood concentration of active retinoid agent in the human or animal of greater than about 100 ng/ml.

36. The method of claim 28 wherein the administering step comprises orally administering a daily dose in a range of about 1 mg to about 6 mg of the retinoid component to the human or animal.

37. The method of claim 28 wherein the administering step comprises orally administering a capsule containing the retinoid component to the human or animal.

38. The method of claim 28 wherein the administering step comprises repeatedly orally administering the retinoid component to the human or animal for a period of time in excess of about 20 weeks.

39. The method of claim 28 wherein the retinoid component includes an active retinoid agent or a precursor of an active retinoid agent effective to more selectively affect at least one of RAR-beta and RAR-gamma relative to RAR-alpha.

40. The method of claim 28 wherein the retinoid component is substantially ineffective to bind with RXRs.

41. The method of claim 28 wherein the retinoid component includes an active retinoid agent more water soluble than isotretinoin or is converted in the human or animal into an active retinoid agent more water soluble than isotretinoin.

42. The method of claim 28 wherein the retinoid component is selected from the group consisting of active acetylenic retinoid agents, precursors of active acetylenic retinoid agents and mixtures thereof.

43. The method of claim 28 wherein the retinoid component is selected from the group consisting of tazarotene, tazarotenic acid and mixtures thereof.

44. The method of claim 28 wherein the retinoid component includes tazarotene.
